(19) United States
(12) Patent Application Publication
(10) Pub. No.: US 2006/0292255 A1
(43) Pub. Date: Dec. 28, 2006

(54) Pharmaceutical and therapeutic compositions derived from Garcinia mangostana L plant

(76) Inventors: Alex Moffett, Chatsworth, CA (US); Parag Shah, Bangalore (IN)

Correspondence Address: GREENBERG TRAURIG, LLP, 1900 UNIVERSITY AVENUE, FIFTH FLOOR, EAST PALO ALTO, CA, 94303, US

(21) Appl. No.: 11/473,486

(22) Filed: Jun. 22, 2006

Related U.S. Application Data
(63) Continuation of application No. 11/281,302, filed on Nov. 16, 2005.
(60) Provisional application No. 60/692,854, filed on Jun. 22, 2005.
(60) Provisional application No. 60/628,615, filed on Nov. 16, 2004.

(51) Int. Cl. A61K 36/185

(52) U.S. Cl. 424/769; 424/777

(57) ABSTRACT

The present invention relates to pharmaceutical, therapeutic, nutritional, cosmetic, and dermatological compositions derived from the preicarp (rind) of the Garcinia mangostana L plant and the novel extraction processes used to produce those compositions. Specifically, the present invention relates, in part, to an approximately 0.01% to about 80% mixture of a xanthone-rich mangosteen pericarp (rind) extract in novel combinations for pharmaceutical, cosmetic, therapeutic or dermatological compositions that yield surprising health benefits. Additionally, the present invention relates, in part, to novel extraction processes that result in the production of the novel compositions of the invention.

### RELATED APPLICATIONS

This patent application claims priority to U.S. Provisional Patent application No. 60/692,854, of Moffett and Shah, filed on Jun. 22, 2005. This patent application is also a continuation of U.S. patent application Ser. No. 11/281,302, and is related to PCT application US2005/041365, by Applicants Parag and Shah, both of which were filed on Nov. 16, 2005.

### FIELD OF THE INVENTION

The present invention relates to pharmaceutical, therapeutic, nutritional, cosmetic and dermatological compositions derived from the preicarp of the Garcinia mangostana L plant and to the novel extraction processes used to produce blended extract compositions.

### BACKGROUND OF THE INVENTION

The mangosteen tree (Garcinia mangostana L.) is a tropical evergreen tree indigenous to the Malay Peninsula, Myanmar, Thailand, Cambodia, Vietnam, the Sunda Islands, and the Moluccas. It is a slow-growing, smooth tree with a pyramidal crown, straight trunk, and flaking black bark that contains a yellow, resinous latex. Its leaves are leathery, shiny, and elliptical. The flowers are unisexual female, thick, fleshy, and green with edges of pinkish red. The mangosteen fruits are smooth, globular berries that ripen to a dark reddish to black-violet color and are normally smooth or marked with brownish scars. Each mangosteen fruit usually varies in weight from 75 to 120 grams and normally contains 2 to 3 well-developed seeds. The pericarp, or rind, of the mangosteen fruit contains pectin and tannins, it is thick, tough, and exudes a bitter yellowish resin. Only about 25 to 30% of the mangosteen fruit consists of the edible pulp, with the remainder is the tough, bitter pericarp or rind.

In contrast to the thick outer pericarp, the edible inner pulp of the mangosteen fruit is widely regarded for its exquisite taste. The inner pulp of a single mangosteen fruit usually consists of four to eight juicy, white-colored segments. When preparing the white pulp segments for consumption, care is particularly taken so as to not stain the pulp segments with the resins, tannins and other matter that oozes out of the pericarp. The need to keep the delicious white pulp separate from the dark purple, staining, bitter pericarp has long been known to those familiar with the mangosteen fruit.

Free radicals are highly reactive chemical species with an odd number of (unpaired) electrons, that are produced in the body. There are several endogenous sources of oxidants: reduction of molecular oxygen in mitochondria during cellular respiration takes place in sequential steps, yielding the radical by-products super oxide O2—, hydroxyl HO, and hydrogen peroxide H₂O₂; degradation of fatty acids and other molecules in peroxisomes produces H₂O₂; phagocytosis results in an oxidative burst of nitric oxide (NO), which also reacts with super oxide to produce the oxidizing and nitrating species peroxynitrite (ONOO⁻).

Free radicals are very unstable and react quickly with other compounds. Once formed, they can start a chain reaction of cell damage, which may ultimately result in death of the cell. For instance, free radicals and oxidants can trigger lipid per oxidation, as well as the oxidation of proteins and DNA, causing extensive damage to body cells. Further, an imbalance of oxidizing species and natural antioxidants in the body leads to oxidative stress, which is believed to contribute to the aging process, cell apoptosis, and severe diseases, such as skin cancer.

Over exposure to the sun contributes to free radical production. The cumulative effects of exposure to ultraviolet radiation (UVA and UVB) are slow to develop, but over the long term they pose serious threats to health, and may become life-threatening. The effects of ultraviolet light on skin include sunburn, photosensitivity, immunosuppression, premature aging, and several types of skin cancer including: premalignant lesions, basal cell carcinoma, squamous cell carcinoma, and malignant melanoma. Other various skin diseases are aggravated or triggered by sunlight exposure; these include various immunologic diseases with cutaneous manifestations, such as lupus erythematosus, solar urticaria, and polymorphous light eruption.

Shorter-wavelength UVB light is absorbed by the vulnerable upper layers of the epidermis causing redness of the skin via direct damage to keratinocytes, and can also lead to the subsequent release of inflammatory mediators. Longer-wavelength UVA penetrates the skin more deeply and is absorbed by melanocytes, elastin, and collagen. UVA contributes relatively little to sunburn erythema, however, the damaging effects of UVA on collagen and elastin are manifested as premature aging and wrinkling of chronically sun-exposed skin. Collagen and elastin fibers lose their resilience over time, become thickened and clumped, and this eventually may lead to dermatoheliosis, epidermal thinning, irregular pigmentation, and telangiectasia.

There are relatively few options for effective protection from UV exposure and the resulting damage it causes. These options include sun avoidance, sun-protective clothing and sunglasses, chemical sunscreens (which act by chemical absorption of UV light), and products containing micronized titanium dioxide or zinc oxide. Although these options can work reasonably well if applied appropriately and consistently none of them fully protect against the oxidative effects of UV damage.

Antioxidants, such as vitamins A, C, E, and selenium, are chemicals found in whole foods (especially fruits and vegetables) that help to protect the body's cells from the harmful effects of free radicals. Xanthones are also natural antioxidants that work at the molecular level, and a rich source of xanthones has been found in the fruit pulp of mangosteen plants of the Garcinia genus (Garcinia mangostana L). In areas where it is indigenous, the mangosteen plant is put to use by people in a variety of ways. The timber is used for building materials and furniture. The rind, or pericarp, is used in the tanning and dyeing industries. The fruit pulp is used as a food product. The rind, leaves and bark are used as ingredients in folk medicine for treating catarrh, cystitis, chronic diarrhea and ulcers, dysentery, eczema, fever, intestinal problems, infected wounds, and skin ailments. The leaves are used in teas and other decoctions for diarrhea, dysentery, fever, and thrush. Recently the whole of the mangosteen plant was formulated into a fruit juice to be taken as a health supplement for the prevention of several various maladies.

A variety of xanthones have been isolated from the mangosteen hull and rinds. In particular, two xanthones, alpha- and gamma-mangostin, were isolated together with (−)-eepicatechin, procyanidins A-2 and B-2 (Yoshikawa et al., 1994, “Antioxidant constituents from the fruit hulls of mangosteen (Garcinia mangostana L.) originating in Vietnam”, Yakugaku Zasshi. 114(2):129-133: in Japanese with English abstr). Mangostanol shows strong inhibition of cAMP phosphodiesterase (Chairungsrilerd and Takeuchi et al., 1996, “Mangostanol, a prenyl xanthone from Garcinia mangostana” Phytochemistry. 43(5): 1099-1102), and gamma-mangostin shows more potent antioxidative activity than BHA (butylated hydroxyanisole, an antioxidant widely used in the food industry), and alpha-tocopherol (vitamin E) (Yoshikawa et al., 1994, as above; and Fan and Su, 1997 “Antioxidative mechanism of isolated components from alcohol extract of fruit hulls of Garcinia mongostana L”. J Chin Agric Chem Soc. 35(5):540-551, in Chinese with English abstr). In fact, gamma-mangostin was found to directly inhibit the activity of cyclooxygenases COX 1 and COX 2 (Nakatani et al., 2002, “Inhibition of cyclooxygenase and prostaglandin E2 synthesis by α-mangostin, a xanthone derivative in mangosteen, in C6 rat glioma cells. Biochem Pharmacol. 63:73-79, and Nakatani et al., 2004, “g-Mangostin inhibits inhibitor-_{K}B kinase activity and decreases lipopolysaccharide-induced cyclooxygenase-2 gene expression in C6 rat glioma cells” Mol Pharmacol. 66(3):667-674.), enzymes that catalyze the first step in the creation of prostaglandins from a common fatty acid by adding two oxygen molecules to arachidonic acid, thus initiating a set of reactions that ultimately creates a host of free radicals. Gamma-mangostin also inhibits DNA topoisomerase (Tosa et al., 1997, “Inhibitory activity of xanthone derivatives isolated from some Guttiferaeous plants against DNA topoisomerases 1 and II” Chem Pharm Bull. 45(2):418-420.) and is an antagonist of serotonin receptors (Chairungsrilerd, Furukawa et al., 1996 “Histaminergic and serotonergic receptor-blocking substances from the medicinal plant Garcinia mangostana. Planta Med. 62(5):471-472 (letter)”, Chairungsrilerd, Furukawa, Ohta et al., 1998, “α-Mangostin, a novel type of 5-hydroxytryptamine 2A receptor antagonist Naunyn-Schmiedeberg's Arch Pharmacol. 357:25-31; Chairungsrilerd, Furukawa, Tadao et al., 1998, “Effect of α-mangostin through the inhibition of 5-hydroxy-tryptamine 2A receptors in 5-fluoro-□-methyltryptamine-induced head-twitch responses of mice” Br J Pharmacol. 123(5):855-862).

There exists a need in the pharmaceutical, therapeutic, cosmetic, dermatological, and sun-protective arts for xanthone-rich compositions that provide effective protection from the short-term burning, long-term photoaging and photocarcinogenesis, and systemic oxidative stress caused by UV damage. Heretofore, the mangosteen rind has not been exploited as a source of xanthone-rich, antioxidant compositions, nor have appropriate extraction processes been available.

### SUMMARY OF THE INVENTION

The described invention provides an anti-oxidative composition, comprising highly concentrated xanthone components extracted from the pericarp or rind of the mangosteen plant, Garcinia mangostana, a rich source of natural xanthones, that can be applied topically for the treatment of a variety of human ailments and conditions in an efficacious manner. The mangosteen plant is also known by various common names, and names particular to different languages. The fruit, capped by the prominent calyx at the stem end and with 4 to 8 triangular, flat remnants of the stigma in a rosette at the apex, is round, dark-purple to red-purple and smooth externally; 3.4-7.5 cm in diameter. The rind is 6-10 mm thick, red in cross-section, purplish-white on the inside; it contains bitter yellow latex and a purple, staining juice. There are 4 to 8 triangular segments of snow-white, juicy, soft flesh (actually the arils of the seeds). The fruit may be seedless or have 1 to 5 fully developed seeds, ovoid-oblong, somewhat flattened, 2.5 cm long and 1.6 cm wide, that cling to the flesh.

In the course of the inventors' search for natural antioxidants, they found a pericarp extract product derived from the fruit hulls of the mangosteen plant (Garcinia mangostana L.) to have a potent radical-scavenging effect. Specifically, compositions comprising a concentrated extract product of the mangosteen pericarp that comprises about 0.1% to about 80%, particularly, concentrations between about 0.3% to about 60%, and more particularly, concentrations of about 1% to about 40% of the total weight of a composition mixture are described. Further, specific embodiments of about 1%, of about 10%, of about 20%, and of about 40% concentrations are described. These embodiments of mixtures are herein shown to possess surprising antioxidant properties, and to be less cytotoxic than previously available crude alcoholic extracts. The medical conditions for which this product is therapeutically useful include sunburn, photosensitivity, immunosuppression, premature aging, psoriasis, several types of skin cancer and various immunologic diseases, as well as inflammation, various bacterial or fungal infections, skin rashes, and oxidative stresses caused by UV radiation exposure and diet.

The present invention relates to pharmaceutical, therapeutic, cosmetic and dermatological compositions derived from the preicarp of the Garcinia mangostana L (mangosteen) plant. A first object of the present invention is to provide pharmaceutical, cosmetic, nutritional, therapeutic, and dermatological compositions that are rich in natural xanthones, are easy to produce and formulate, and which benefit human health by, for example, counteracting the cancerous and aging effects of photo-oxidation caused by exposure to ultraviolet (UV) radiation of the three classes, based on wavelength, UVA (320-400 nM), UVB (280-320 nM), and UVC (less than 280 nM) radiation. Accordingly, presented herein are novel compositions comprising about a 0.1% to about an 80% xanthone-rich concentrate mixture derived from a novel extraction process of the mangosteen pericarp/rind that yields surprising anti-oxidative health benefits. In addition to their powerful antioxidant effects, the compositions of the present invention also show strong antiseptic, antibacterial, and antiviral effects, are supportive of the immune response and wound healing, and are easy to produce and formulate.

Accordingly, in embodiments of the present invention, a xanthone-rich mangosteen pericarp extract is present in a composition in an amount ranging from between about 0.1% to about 80%, particularly between about 0.3% to about 60%, and most particularly between about 1% and about 40% of the total weight of the composition mixture; additionally, specific embodiments include compositions of about 1%, 10%, 20%, and 40%. Such compositions comprise at least one, and generally an abundant plurality of the following xanthones: calabaxanthone, demethylcalabaxanthone, 6-deoxy-γ-mangostin, 1-isomangostin, 3-isomangostin, 1-isomangostin hydrate, 3-isomangostin hydrate (Mahabusarakam et al., 1987), gartanin (Chairungsrilerd et al., 1996), 8-deoxygartanin (Chairungsrilerd et al., 1996; Govindachari et al., 1971; Sakai et al., 1993), garcinone A (Sen et al., 1982), garcinone B (Sakai et al., 1993), garcinone C (Sen et al., 1982), garcinone D (Sen et al., 1986), garcinone E, mangostanol (prenyl xanthone), mangostanol (polyoxygenated xanthone), α-mangostin (Chairungsrilerd et al., 1996), β-mangostin (Govindachari et al., 1971 b; Sakai et al., 1993), γ-mangostin (Chairungsrilerd et al., 1996), 6-deoxy-γ-mangostin (Sakai et al., 1993), mangostinone 1,5-dihydroxy-2-(3-methylbut-2-enyl)-3-methoxyxanthone, 1,7-dihydroxy-2-(3-methylbut-2-enyl)-3-methoxyxanthone (Asai et al., 1995), 1,5-dihydroxy-3-methoxy-2-(3-methylbut-2-enyl)xanthone (Sen et al., 1981), 1,7-dihydroxy-3-methoxy-2-(3-methylbut-2-enyl)xanthone (Mahabusarakam et al., 1987; Sen et al., 1981), 5,9-dihydroxy-2,2-dimethyl-8-methoxy-7-(3-methylbut-2-enyl)-2H,6H-pyrano[3,2b]xanthen-6-one, 2-(γ,γ-dimethylallyl)-1,7-dihydroxy-3-methoxyxanthone (Chairungsrilerd et al., 1996), 2,7-di-(3-methylbut-2-enyl)-1,3,8-trihydroxy-4-methylxanthone, 2,8-di-(3-methylbut-2-enyl)-7-carboxy-1,3-dihydroxyxanthone (Gopalakrishnan and Balaganesan, 2000), normangostin (ν-mangostin) (Govindachari et al., 1971), 1,5,8-trihydroxy-3-methoxy-2-(3-methyl-2-butenyl)xanthone, 1,7-dihydroxy-2-isoprenyl-3-methoxyxanthone, xanthone I (Sakai et al., 1993), BR-xanthone A, BR-xanthone B (2,4,5-trihydroxy-1-methoxyxanthone) (Balasubramanian and Rajagopalan, 1988), garcinone B, mangostanol, mangostenol, mangostenone A, mangostenone B, α-mangostin, β-mangostin, mangostinone, tovophyllin, and trapezifolixanthone (Suksamrarn et al., 2002). Compositions containing such listed components may further include any and all active phytochemicals existing in the rind or a combination thereof. Particular embodiments of the about 0.1% to about 80% xanthone-rich extract includes at least one of the following xanthones: alpha-, beta-, and gamma-mangostins, as well as (−)-epicatechin, procyanidins A-2 and B-2, garcinones A to E, maclurin or a combination mixture of any of the above. In other more particular embodiments, the xanthone-rich extract includes mangostins; more particularly still, the xanthone-rich extract includes alpha-, beta or gamma-mangostins, or a combination thereof.

The therapeutic effectiveness of these compositions may further be heightened by the addition of other selected pharmaceutical, therapeutic, cosmetic, and dermatological ingredients in varying amounts (such ingredients may also be referred to as “second agents”, see the fourth object of the invention below). These ingredients may include preservatives, treatment agents (such as antimicrobial, anti-fungal, and anti-inflammatory agents), vitamins, flavonoids, solvents, surfactants, emulsifying agents, humectants, fragrances, and the like.

A second object of the present invention is to provide a process for preparing pharmaceutical, therapeutic, nutritional, cosmetic, and/or dermatological compositions derived from the Garcinia mangostana L. plant that are rich in natural xanthones, and which yield health benefits of the mangosteen pericarp/rind, either alone or with other complementary and enhancing constituents. Accordingly, in another embodiment of the present invention, a practical and economical process for manufacturing pharmaceutical, therapeutic, cosmetic, and/or dermatological compositions derived from the pericarp/rind of the Garcinia mangostana L. plant is provided. The process results in a xanthone-rich extract product that is between about 0.1% to about 80%, particularly between about 0.3% to about 60%, and more particularly between about 1% to about 40% xanthones. In some embodiments, the about 0.1% to about 80% xanthone-rich extract comprises a xanthone from the list set forth above, but more particularly includes at least one of the following xanthones: alpha-, beta- or gamma-mangostins, as well as (−)-epicatechin, procyanidins A-2 and B-2, or a combination mixture thereof. More particularly, the xanthone-rich extract includes mangostins. More particularly still, the xanthone-rich extract includes alpha-mangostins and/or gamma-mangostins.

The inventive processes for the preparation of these varying compositions make use of the preparation of highly concentrated extracts and less concentrated extracts, and their blending or admixing to obtain final desired concentrations. Further, tannins are reduced in concentration in the xanthone-rich extracts, relative to the initial, naturally occurring relative concentrations by these processes. By dissociating the relative degree of concentrating of xanthones and tannins, respectively, the concentration of tannins can be modulated independently of the concentration of xanthones. By independently adjusting or modulating the tannin concentration, the antioxidant value of tannins can be exploited fully, while avoiding the unwanted potentially-cytotoxic effects of tannin that may manifest at high concentrations.

A third object of the present invention is to provide methods of treating or providing prophylactic measures for diseases and conditions of skin that result from exposure to sunlight, and which may be mediated by the generation of reactive oxygen species during such sunlight exposure. A fourth object of the invention is to provide therapeutic compositions that combine the xanthone-rich extract of Garcinia mangostana with other second agents which have therapeutic or cosmetic benefit for the skin, thereby enhancing the efficiency and benefit that each agent would provide alone.

The foregoing and other objects, advantages, and characterizing features of embodiments of the invention will become apparent from the description of illustrative embodiments that follows, and in the appended claims. While the formulations of the present invention have proven to be particularly useful in the area of pharmaceutical, therapeutic, cosmetic, and dermatological compositions, those skilled in the art can appreciate that such formulations and mixtures can be used in a variety of different applications and in a variety of different types of manufacture to satisfy a wide-ranging variety of pharmaceutical and medicinal needs. Further, the features and advantages of the invention may be learned by the practice of the invention, or will be obvious to one skilled in the art from reading the description, as set forth hereinafter.

### BRIEF DESCRIPTION OF THE FIGURES

FIG. 1 is a flowchart for process A.1, a water extraction process.

FIG. 2 is a flowchart for process A.2, an alcohol extraction process.

FIG. 3 is a flowchart for process A.3, an additional water extraction process.

FIG. 4 is a flowchart for process A.4, an enrichment process.

FIG. 5 is a flowchart for process B.1, an extraction process with an organic solvent.

FIG. 6 is a flowchart for process B.2, a water extraction process.

FIG. 7 is a flowchart for process B.3, an enrichment process.

FIG. 8 is a flowchart for the process for mangostin extract of 1%.

FIG. 9 is a flowchart for the process for mangostin extract of 20%.

FIG. 10 is a flowchart for process for mangostin extract of 40%.

FIG. 11 is a flowchart for process for mangostin extract of 40%, method 1.

FIG. 12 is a flowchart for process mangostin extract of 40%, method 2, process 1.

FIG. 13 is a flowchart for process mangostin extract of 40%, method 2, process 2.

### DETAILED DESCRIPTION OF THE INVENTION

Pharmaceutical, Therapeutic, Nutritional, Cosmetic, and Dermatological Compositions Active Ingredients

The present invention relates to pharmaceutical, therapeutic, nutritional, cosmetic, and/or dermatological compositions derived from the Garcinia mangostana L. plant (the mangosteen plant). “Mangosteen” is term that refers generally to the plant, and is also used as an adjective, as in “mangosteen pericarp”, “mangosteen extract”, “mangosteen compounds”, or “mangosteen compositions”, such latter two terms referring to extracts of the plant, compounds produced by the plant, and to compositions comprising such compounds. Embodiments of the invention described herein uniquely provide natural compounds, generally xanthones, extracted from the pericarp of the mangosteen plant; more particularly, it is disclosed that an approximately 0.1% to about 80% xanthone concentrate extracted from the mangosteen pericarp (rind) and formulated into a pharmaceutical, cosmetic, therapeutic, or dermatological composition yields benefits to aspects of human health. In embodiments of the present invention, the xanthone-rich mixture of mangosteen pericarp extract is present in an amount ranging from between about 0.1% to about 80%, particularly between about 0.3% to about 60%, and more particularly between about 1% about 40% of the total weight of composition mixture. Other specific embodiments include extract compositions of about 1%, of about 10%, of about 20%, and of about 40% of the total weight of the composition.

The xanthone-rich extract comprises at least one of the following xanthones: calabaxanthone, demethylcalabaxanthone, 6-deoxy-γ-mangostin, 1-isomangostin, 3-isomangostin, 1-isomangostin hydrate, 3-isomangostin hydrate, gartanin, 8-deoxygartanin, garcinone A, garcinone B, garcinone C, garcinone D, garcinone E, mangostanol (prenyl xanthone), mangostanol (polyoxygenated xanthone), α-mangostin, β-mangostin, γ-mangostin, mangostinone, 1,5-dihydroxy-2-(3-methylbut-2-enyl)-3-methoxyxanthone, 1,7-dihydroxy-2-(3-methylbut-2-enyl)-3-methoxyxanthone, 1,5-dihydroxy-3-methoxy-2-(3-methylbut-2-enyl)xanthone, 1,7-dihydroxy-3-methoxy-2-(3-methylbut-2-enyl)xanthone, 5,9-dihydroxy-2,2-dimethyl-8-methoxy-7-(3-methylbut-2-enyl)-2H,6H-pyrano[3,2b]xanthen-6-one, 2-(γ, γ-dimethylallyl)-1,7-dihydroxy-3-methoxyxanthone, 2,7-di-(3-methylbut-2-enyl)-1,3,8-trihydroxy-4-methylxanthone, 2,8-Di-(3-methylbut-2-enyl)-7-carboxy-1,3-dihydroxyxanthone, normangostin (ν-mangostin), 1,5,8-trihydroxy-3-methoxy-2-(3-methyl-2-butenyl)xanthone, 1,7-dihydroxy-2-isoprenyl-3-methoxyxanthone, xanthone 1, BR-xanthone A, BR-xanthone B (2,4,5-trihydroxy-1 -methoxyxanthone), garcinone B, mangostanol, mangostenol, mangostenone A, mangostenone B, tovophyllin, and trapezifolixanthone, and may include any and all active phytochemicals existing in the rind, or a combination thereof. In a more summary form of accounting, particular embodiments of the invention, the (about 0.1% to about 80%) xanthone-rich extract includes at least one of the following three xanthones: alpha-, beta-, and gamma-mangostins (α-, β-, γ-), in any combination and relative proportion; other particular embodiments may further include any of (−)-epicatechin, procyanidins A-2 and B-2, garcinones A-E, maclurin, and still other embodiments may further include any and all active phytochemicals extant in the rind, or a combination mixture of any of the preceding compounds. The question as to the relative abundance of the three mangostin forms (α-, β-, γ-) in Garcinia plants is still unsettled among those who know the field; the inventors are of the belief that α-mangostin is the generally the most naturally abundant, and thus the one with the greater relative presence in embodiments of this invention However, neither the invention nor the claims are bound by this belief, and reference to “mangostin” or “mangostins” refers to the collective presence of all forms of mangostin. Further, other embodiments of the pharmaceutical, therapeutic, cosmetic, and/or dermatological compositions include the full range of compounds and compositions just described without the limitation of being immediately sourced from the Garcinia mangostana plant. Accordingly such embodiments could include natural-sourced compounds derived, from other plants or organisms, or compounds derived by way of synthetic production methods, or from genetically engineered organisms.

The effectiveness of these therapeutic mixtures can further be heightened through the addition of other selected pharmaceutical, therapeutic, cosmetic, and dermatological formulation ingredients in varying amounts. Such ingredients may, by way of example, include preservatives, treatment agents (such as antimicrobial, anti-fungal, and anti-inflammatory agents), vitamins, flavonoids, solvents, surfactants, emulsifying agents, humectants, fragrances and the like. Further description of so-called second agents is expanded on in a following section, below.

Inclusion of Second Active Agents in the Composition

The compositions set-forth here, in accordance with embodiments of the invention, may take various forms and be used for various topical applications, such as, by way of example, lotions, ointments, gels, foams, or bars, and variously for moisturizing, cleansing or disinfecting lotions, and may include anti-fungal or bactericidal agents. Typically the compositions of the invention constitute additionally protective, treatment, or care creams, lotions, gels, foams, or soaps for the body, in particular for the skin of various body parts, including the face, the limbs, the hands, the feet, for the major anatomical folds of the body and/or the mucous membranes. Accordingly, in addition to a mangosteen pericarp extract component, these compositions may include one or more other active agents, or second therapeutic agents, for preventing and/or treating other various skin complaints, conditions, and/or afflictions. “Second agent” in this context refers to therapeutic agents other than those comprising the Garcinia mangostana extract, and is a general term that even when used in the singular may refer to a one or more such agents. Adverse skin conditions treatable by embodiments of the present invention, particularly with the inclusion of second agents, may include, merely by way of example, conditions of abnormal cutaneous differentiation, proliferation, or pigmentation, bacterial infections, parasitic infections, fungal infections, inflammation, pain or irritation from any source, pruritis, viral agents, keratolysis, UV radiation damage, seborrhea, dandruff, or acne. Agents that treat such various adverse skin conditions, accordingly, may include modifiers of cutaneous differentiation and/or proliferation and/or pigmentation, antibacterial agents, antiparasitic agents, antifungal agents, steroidal anti-inflammatory agents, anaesthetic agents, antipruriginous agents, antiviral agents, keratolytic agents, other anti-oxidants, antiseborrhoeic agents, antidandruff agents, and antiacne agents.

Examples of these active second agents, summarized below in Table 1, include: (1) agents that modify cutaneous differentiation and/or proliferation and/or pigmentation such as, by way of non-exclusive example, retinoic acid and isomers thereof, retinol and esters thereof, vitamin E and D and derivatives thereof, estrogens such as estradiol, kojic acid or hydroquinone; (2) antibacterial agents such as, by way of non-exclusive example, clindamycin phosphate, erythromycin or antibiotics from the tetracycline family; (3) antiparasitic agents, such as, by way of non-exclusive example, metronidazole, crotamiton or pyrethroids; (4) antifungal agents, such as, by way of non-exclusive example, compounds of the imidazole family such as econazole, ketoconazole or miconazole or salts thereof, polyene compounds such as amphotericin B, compounds of the allylamine family, such as terbinafine, or alternatively octopirox; (5) steroidal anti-inflammatory agents such as, by way of non-exclusive example, hydrocortisone, betamethasone valerate or clobetasol propionate, or nonsteroidal anti-inflammatory agents such as ibuprofen and salts thereof, diclofenac and salts thereof, acetylsalicylic acid, acetaminophen or glycyrrhetinic acid; (6) anaesthetic agents such as, by way of non-exclusive example, lidocaine hydrochloride and derivatives thereof; (7) antipruriginous agents such as, by way of non-exclusive example, thenaldine, trimeprazine or cyproheptadine; (8) antiviral agents such as acyclovir; (9) keratolytic agents such as, by way of non-exclusive example, alpha- and beta-hydroxycarboxylic acids or beta-ketocarboxylic acids, salts, amides or esters thereof and more particularly hydroxy acids such as glycolic acid, lactic acid, salicylic acid, citric acid and fruit acids in general, and 5-n-octanoylsalicylic acid; (10) other anti-free-radical-agents, or anti-oxidants such as, by way of non-exclusive example, alpha-tocopherol or esters thereof, superoxide dismutases, certain metal-chelating agents or ascorbic acid and esters thereof; (11) antiseborrhoeic agents such as, by way of non-exclusive example, progesterone; (12) antidandruff agents such as, by way of non-exclusive example, octopirox or zinc pyrithione; and (13) antiacne agents such as retinoic acid or benzoyl peroxide. Further included may be natural-sourced components that are considered to provide healthful or ameliorative benefits, such as pomegranite, green tea, ale, turmeric extracts, seaweed-derived peptides and proteins, and various minerals, both simple and complex.

TABLE 1
Second Active Agents

| Class of Agent | Examples |
|---|---|
| Modifiers of cutaneous | retinoic acid and isomers thereof, retinol and esters thereof, vitamin E |
| differentiation, | and D and derivatives thereof, estrogens such as estradiol, kojic acid, |
| proliferation, or | or hydroquinone |
| pigmentation | |
| Antibacterial | clindamycin phosphate, erythromycin or antibiotics from the |
| | tetracycline family |
| Antiparasitic | metronidazole, crotamiton or pyrethroids |
| Antifungal | compounds of the imidazole family such as econazole, ketoconazole |
| | or miconazole or salts thereof, polyene compounds such as |
| | amphotericin B, compounds of the allylamine family, such as |
| | terbinafine, or octopirox |
| Steroidal anti- | hydrocortisone, betamethasone valerate or clobetasol propionate, or |
| inflammatory | nonsteroidal anti-inflammatory agents such as ibuprofen and salts |
| | thereof, diclofenac and salts thereof, acetylsalicylic acid, |
| | acetaminophen or glycyrrhetinic acid |
| Anaesthetic | lidocaine hydrochloride and derivatives thereof |
| Antipruriginic | thenaldine, trimeprazine or cyproheptadine; |
| Antiviral | acyclovir |
| Antikeratolytic | alpha- and beta-hydroxycarboxylic acids or beta-ketocarboxylic acids, |
| | salts, amides or esters thereof and more particularly hydroxy acids |
| | such as glycolic acid, lactic acid, salicylic acid, citric acid and fruit |
| | acids in general, and 5-n-octanoylsalicylic acid |
| Other anti-oxidants | alpha-tocopherol or esters thereof, superoxide dismutases, certain |
| | metal-chelating agents or ascorbic acid and esters thereof |
| Antiseborrhoeic | progesterone |
| Antidandruff | octopirox or zinc pyrithione |
| Antiacne | retinoic acid or benzoyl peroxide |

Medical Indications

The medical conditions or indications for which this product is therapeutically efficacious include conditions that result from exposure to sunlight, more specifically to the ultraviolet radiation of type UVA, UVB, and UVC, as well as the damage more specifically associated with exposure to reactive oxygen species, whatever their source. Embodiments of therapeutic compositions described herein are understood to be therapeutically efficacious when they are useful for the prevention of disease or when they are useful for treatment of extant disease, where treatment may include amelioration of symptoms, slowing of progression of disease, or cure of disease. Conditions directly or indirectly a consequence of (or are exacerbated by, or include as a risk factor) exposure to such radiation and reactive oxygen species include both direct and immediate effects, as well as longer term effects, and complications and sequellae that arise from the direct damage, over a longer term. Inasmuch as embodiments act prophylactically, or treat incipient disease at a very early stage, embodiments of the invention are bioprotective, or more specifically, for example, cardioprotective. Embodiments of the invention are thus bioprotectants, whose bioprotective effects in organs and tissues may manifest in specific ways, as for example, prevention of cellular apoptosis, or intervention in allergic or inflammatory processes, whether localized or widespread.

Generally, health problems associated with exposure to ultraviolet radiation involved conditions or diseases of the skin, but more widespread and systemic conditions may also arise, or be a part of complications that follow on as a consequence of such conditions or diseases of the skin. Accordingly, such conditions, collectively, may include sunburn, photosensitivity, immunosuppression, premature aging, psoriasis, several types of skin cancer and various immunologic diseases, as well as localized or widespread inflammation, various bacterial or fungal infections, skin rashes, and systemic oxidative stresses caused by UV radiation exposure and diet. Actinic keratosis, for example, are precancerous lesions that develop after many years of sun exposure, polymorphic light eruption (PMLE), for example, is a rash induced by sunlight exposure, which is understood as involving skin-localized allergy and the immune system. Types of skin cancer linked to sunlight exposure include, in order of increasing seriousness, basal cell cancer, squamous cell cancer, and malignant melanoma.

Though not bound by theory, the inventors theorize that ultraviolet light impacts skin through both direct and indirect mechanisms. The direct damage is that which is incurred upon immediate exposure to radiation, the indirect effects include those which follow the generation of damaged biological molecules and the generation of highly reactive oxygen species (ROS), which then set other biological and pathological processes in motion. The reactive oxygen species may have deleterious effects in the immediate locale where they are generated, as in the skin, or at distant sites, where such reactive species may have broader systematic effects, as may manifest in what is termed “oxidative stress”. An intervention that effectively reduces the level of reactive species, thereby having an anti-oxidant effect, thus may have slow, ameliorate, or stop the progression of a broad range of diseases. Further, inventors theorize that effectiveness of the delivery of an anti-oxidant therapeutic agent to cells exposed to reactive oxygen species may be important in the clinical success of the agent. Accordingly, formulation and administration aspects of embodiments of the invention are described below.

Formulations and Routes of Administration

The compositions, according to embodiments of the invention, may comprise all pharmaceutical forms normally utilized according to the route of administration (e.g., topical, injection, or oral route) to achieve the therapeutic effect desired. Readily flowable forms such as solutions and micro-emulsions may also be employed for example, for intralesional injection (for the treatment of various skin maladies) or for rectal administration, e.g., as an enema for the treatment of inflammatory bowel disease, Crohn's disease, ulcerative colitis, or the like. Compositions in accordance with the invention, however, are typically intended for oral or topical application, in particular application to the skin.

Accordingly, compositions representing embodiments of the present invention may be employed for administration in any appropriate manner, e.g., topically, for instance, for application to the skin, for example in the form of a cream, paste, lotion, gel, ointment, poultice, cataplasm, plaster, dermal patch or the like, orally for instance in unit dosage form (e.g., in hard or soft gelatin encapsulated or tablet form), or parenterally. Additionally, the compositions of the invention may be utilized in conjunction with advanced topical delivery technologies, such as lipoid or liposomal technologies that make use of self-assembled lipid structures, to control the release rate and depth of active ingredient penetration without greatly disturbing the skin barrier. Such compositions may include, by way of example, phosphatylcholines, ceramides (I, III, & VI), cholesterol, palmitic acid, mevalonic acid, glycerol, and/or 25-hydroxycholecalciferol or any mixture thereof, formulated into a differentiated micro carrier system that can be a self-assembling, balanced lipid matrix that is particularly amenable to delivery in the hydro or foam phase, as well as in lipid particles or vesicles.

For topical, oral, and/or parenteral applications, the subject compositions of embodiments of the invention may be formulated into any pharmaceutical form normally employed for such an application, in particular in the form of aqueous, aqueous/alcoholic or oily solutions, dispersions of lotion or serum type, anhydrous or lipophilic gels, emulsions of liquid or semi-liquid consistency, for instance of the milk type, obtained by dispersion of a fatty phase in an aqueous phase (oil in water, o/w) or conversely (water in oil, w/o), or suspensions or emulsions of runny, semi-solid, or solid consistency of the cream, gel, or foam type, or alternatively microemulsions, microcapsules, microparticles, or vesicle dispersions of ionic and/or nonionic type, or even powders, or alternatively in the form of aerosol compositions also containing a propellant under pressure. These compositions are formulated according to conventional techniques well known in the art.

For topical administration, typically, the pharmaceutical, cosmetic, therapeutic and/or dermatological compositions of embodiments of the invention comprise a unique cream base composition that is suitable for use alone, as an emollient cream, or in combination with one or more additional cosmetic or dermatological ingredients. Such additional ingredients include, merely by way of example, preservatives, treatment agents, humectants, and fragrances. If desired, the cosmetic cream base composition of the present invention is used in conjunction with a microencapsulated fragrance to form a fragrancing cream composition that can be applied directly to the skin of the wearer. Compositions for oral administration may be formulated as drinkable liquids, wafer capsules, gelatin capsules, syrups, or tablets. For instance, the subject compositions may be administered in the form of aqueous, alcoholic or aqueous/alcoholic solutions in the appropriate surfactants and/or solvents.

Accordingly, in the case where the compositions of the invention are intended for either topical or oral administration, a variety of other components may be added to the mangosteen extract component to make up the final net weight composition. For instance, pharmaceutically acceptable surfactants, solvents, thickening agents (for sustained release), or a combination thereof may also be included. Exemplary solvents, according to the present invention, include the lower alcohols, such as, by way of example, ethanol and isopropanol, as well as most other alkyl alcohols such as butyl alcohol and propylene glycol, and the like. Examples of suitable lipophilic surfactants may include, e.g., trans-esterification products of natural vegetable oil triglycerides and polyalkylene polyols that are known in the art. Such oils may include transesterification products of various natural (e.g. non-hydrogenated) vegetable oils for example, maize oil, kernel oil, almond oil, ground nut oil, olive oil and palm oil and mixtures thereof with polyethylene glycols, in particular polyethylene glycols having an average molecular weight of from 200 to 800 Daltons. Hydrophilic surfactants, particularly non-ionic hydrophilic surfactants, may also be included. Suitable hydrophilic surfactant components are any of those well known in the art and hereinbefore described.

In particular, compositions of the invention that comprise a surfactant or both a surfactant and a solvent (co-solvent), may be formulated in various ways that are known in the art, for example, emulsions, emulsion pre-concentrates (i.e., compositions which, on contacting with water, provide regular emulsions), microemulsions of the o/w or w/o type, emulsions of both hydrophilic/lipophilic and lipophilic/hydrophilic type, and other forms such as solutions, suspensions, dispersions, and the like.

In the case of emulsion pre-concentrates, o/w emulsions as such may be appropriate, in particular where oral administration is contemplated. In the case of formulations, e.g., for drinking or for topical application, they will in particular include aqueous emulsions of o/w or w/o type. Exemplary emulsifying agents, for example, may include glyceryl stearate, polysorbate 60, and/or a mixture of PEG-6/PEG-32/glycol stearate.

When the compositions of the invention are formulated as an emulsion, the proportion of the fatty phase relative to the total weight of the composition should be such to achieve an advantageous range so as to enable maximum absorption of the mangosteen pericarp extract. Hence, the emulsion may also contain various oils and lipid vesicles. The oils, the emulsifying agents, and the co-emulsifying agents employed in the compositions in emulsion form are selected from among those used conventionally in the cosmetic and dermatological fields.

Exemplary oils which are suitable for the compositions of the invention include mineral oils (liquid petrolatum), plant oils (liquid fraction of karite butter and sunflower oil), animal oils (perhydrosqualene), synthetic oils (purcellin oil), silicone oils (cyclomethicone), and fluoro oils (perfluoropolyethers). Fatty alcohols, fatty acids (stearic acid) and waxes (paraffin wax, carnauba wax or beeswax) may also be used as fats. When the compositions of the invention comprise an oily gel or solution, such as for topical application, the fatty phase may constitute a greater proportion of the total weight of the composition.

When the compositions of the invention comprise a liquid emollient component in combination with the mangosteen extract component, the liquid emollient component is typically present at about 5 to about 10 weight percent, and more particularly at about 8 weight percent. The liquid emollient component in topical formulations typically includes C12-15 alcohol benzoate, so as to make a non-greasy emollient or combination of emollients that are nonirritating to the eyes and skin. The topical emollients typically impart a dry lubricating feel to skin. When the compositions of the invention comprise a solid emollient component in combination with the mangosteen extract component, the solid emollient component typically is present at about 2 to about 10 weight percent. When a liquid emollient is used in combination with a solid emollient, the emollient component is typically present at a weight percent of up to about 20 weight percent. In one embodiment for topical delivery, a composition according to the present invention includes a solid emollient in addition to the mangosteen extract component and may also include a wax component discussed below. In particular, dialkyl fumarate is a solid emollient imparting an elegant, non-greasy feel that is appropriate for use as a solid emollient. For the purposes of the present invention, dialkyl fumarate, and di-C12-15-alkyl fumarate particularly, can be considered a wax, although it is commonly considered an emollient.

The pharmaceutical, therapeutic, cosmetic, and/or dermatological compositions of the invention may also contain purified or non-purified additives and adjuvants common in such fields, such as hydrophilic or lipophilic gelling or active agents, preservatives, flavonoids, additional antioxidants, solvents, fragrances, fillers, sunscreens, bactericides, odor absorbers, dyestuffs, and colorants. Common natural products such as pomegranate, green tea, aloe, and extracts of turmeric, seaweed-derived peptides and protein, as well as minerals may be included as well. The amounts of these various additives and adjuvants are those used conventionally in the fields under consideration and range, for example, from 0.01% to 90% of the total weight of the composition. Depending on their particular nature, these additives and adjuvants may be introduced into the fatty phase, into the aqueous phase, and/or into lipid spherules. Exemplary hydrophilic gelling agents which are suitable include carboxyvinyl polymers (carbomer), acrylic copolymers such as acrylate/alkylacrylate copolymers, polyacrylamides, polysaccharides such as hydroxypropylcellulose, natural gums and clays, and, as lipophilic gelling agents, representative thereof are the modified clays such as bentones, fatty acid metal salts such as aluminum stearates and hydrophobic silica, or alternatively ethylcellulose and polyethylene. Exemplary hydrophilic active agents which may be incorporated include proteins or protein hydrolysates, amino acids, polyols, urea, allantoin, sugars and sugar derivatives, water-soluble vitamins, starch and plant extracts, in particular Aloe vera extracts. Exemplary lipophilic active agents include retinol (vitamin A) and derivatives thereof, tocopherol (vitamin E) and derivatives thereof, essential fatty acids, ceramides and essential oils.

An exemplary cream or lotion base composition of the present invention for topical administration may include a substantial powder component, which may be in combination with a wax component, a volatile component, and/or a fragrance component. A suitable powder component may be selected from one or more of the following powders: corn starch, oat starch and spherical silicone dioxide. The use of starch is appropriate for its appealing, soft texture and smooth finish, as well as for its ability to absorb or adsorb the waxes and fluids of the composition of the present invention. Modified starches that are powders and/or liquids may also be used. However, it is appropriate for the starch or starches used in the compositions of the present invention be processed so they do not have a whitening effect when rubbed on the body. Additionally, spherical silicone dioxide may be included to enhance the feel of the starch component on application to the skin.

When a starch and/or other powder is included in the composition, it is appropriate that it be present in a cream base composition of the invention at about 20 to about 90 weight percent of the composition. In a fragrant composition containing fragrant microcapsules, the starch and other powders are typically about 20 to about 60 weight percent of the total weight of the composition, where the weight percent of the starch component is typically adjusted to accommodate the powdery particulate fragrance microcapsules when they are present.

When a wax component is included in the pharmaceutical, therapeutic, cosmetic, and/or dermatological cream base composition of the present invention one or more of the following waxy components are appropriate: ozokerite, myristyl myristate, petrolatum and hydrogenated castor oil. Other waxes will also work in the compositions of the present invention, provided there is at least one microcrystalline wax present for stability. Ozokerite and petrolatum are the typical microcrystalline waxes for use in the present invention. When wax is present in the cream base composition of the present invention it is typically at about 10 to about 30 weight percent of the total weight of the composition.

A volatile component may also be included to add a silky finish to the cosmetic cream base composition when it is applied to the body. Typically, this volatile component may include cyclomethicone, isoeicosane, or a combination of the two. Other volatile components would function in the compositions of the present invention as long as they are able to be processed at the temperatures needed to melt waxes, e.g. at about 170 to about 180° F., without flashing off. The volatile component, if included, is typically present at about 5 to about 20 weight percent.

Where topical application is foreseen, thickening agents may also be included. Suitable thickening agents may be of those known and employed in the art, including, e.g., pharmaceutically acceptable polymeric materials and inorganic thickening agents, for example of the following types: polyacrylate and polyacrylate co-polymer resins, for example poly-acrylic acid and poly-acrylic acid/methacrylic acid resins; celluloses and cellulose derivatives, including alkyl celluloses, hydroxyalkyl-celluloses, acylated celluloses, and salts thereof such as sodium-carboxymethyl-celluloses; polyvinylpyrrolidones, including for example poly-N-inylpyrrolidones and sinylpyrrolidone co-polymers such as vinylpyrrolidone-vinylacetate co-polymers; polyvinyl resins, e.g. including polyvinylacetates and alcohols, as well as other polymeric materials including gum traganth, gum arabicum, alginates, e.g. alginic acid, and salts thereof, e.g. sodium alginates. Inorganic thickening agents such as atapulgite, bentonite, and silicates including hydrophilic silicon dioxide products may also be used.

The compositions of the invention may also include one or more further ingredients in particular diluents, anti-oxidants e.g., ascorbyl palmitate, butyl hydroxy anisole (BHA), butyl hydroxy toluene (BHT) and tocopherols, e.g., α-tocopherol (vitamin E), flavoring agents (purified flavonoids) and so forth, as mentioned above. Use of an anti-oxidant, such as a tocopherol in addition to the mangosteen extract component, is particularly advantageous. Similarly, where the compositions comprise a hydrocolloid thickening agent the composition may also include water, thus providing an aqueous micro-emulsion in gel, paste, cream or like form.

Furthermore, as mentioned, the pharmaceutical, cosmetic, therapeutic and/or dermatological cream base composition of the present invention may also include a fragrance. Typically, the fragrance includes either a microencapsulated fragrance or a non-microencapsulated fragrance or both. If a fragrance is included, typically it is at about 0.01 to about 20 weight percent. While the typical microencapsulated fragrances have a powdery consistency, they are not considered powders when determining preferred powder weight percents for purposes of this invention.

Other optional components may be added to the composition of the present invention. For example, preservatives, treatment agents (e.g., vitamins or ceramides), and humectants (e.g., lactic acid) may be included in the composition to enhance the appearance or the function of the composition. Examples of such additional components suitable for use in accordance with the present invention are those known and commercially available.

Additionally, using combinations of the components described herein the subject compositions may also be formulated as solid preparations constituting soaps or cleansing bars. The injectable compositions may be formulated as an aqueous or oily lotion, or in the form of a serum.

The amounts of the various constituents of the compositions according to the invention are those conventionally used in the fields under consideration and can be formulated into various compositions according to intent by means and in combinations that are well known in the arts. The prophylactic, therapeutic, and/or cosmetic and dermatological treatments according to the invention may be carried out, in particular for topical use, by applying the cosmetic or hygienic compositions to the skin of the body, according to the usual techniques for administering these compositions. For example: application of creams, gels, sera, and lotions to the skin, the scalp and/or the mucous membranes.

However, although the prophylactic, therapeutic, and/or cosmetic and dermatological compositions of the present invention are typically formulated as detailed above, the present compositions may also be delivered in any form known in the art, such as tablets, capsules, dispersions, solutions, suspensions, other transdermal delivery systems, such as lipophilic patches, soaps, or deodorants. If the mangosteen pericarp extract mixture is complemented with juice concentrates, then a liquid beverage is a convenient delivery form, but other delivery forms are equally efficacious and would simply require the use of powders, excipients, adjuvants, or other equivalent forms of carriers. Tablets or capsule forms of the present compositions can be prepared and coated by methods known to those of ordinary skill in the art. The efficacy of this xanthone-rich mixture of mangosteen pericarp extract may also be enhanced through the addition of other ingredients that are believed to synergistically react with the natural xanthone compounds.

Process for the Production of Pharmaceutical, Therapeutic, Nutritional, Cosmetic, and/or Dermatological Compositions from the Mangosteen Pericarp Rind Extract

An object of the present invention is to provide a process for preparing pharmaceutical, therapeutic, cosmetic, and/or dermatological compositions derived from the Garcinia mangostana L. plant that are rich in natural xanthones and thus yield holistic benefits of the mangosteen pericarp/rind, either alone or with other complementary and enhancing constituents, such as flavonoids and/or tannins, or with other “second” therapeutic agents. Accordingly, in another aspect of the present invention, an economical process for manufacturing pharmaceutical, therapeutic, cosmetic, and/or dermatological compositions derived from a pericarp (rind) extract of the Garcinia mangostana L. plant is presented.

The present invention relates specifically to novel processing methods for the extraction of a xanthone-rich extract product from the pericarp/rind of the mangosteen plant that comprises about a 0.1% to about an 80% weight component of a final pharmaceutical, therapeutic, cosmetic, and/or dermatological composition. Typically, the about 0.1% to about 80% xanthone-rich extract comprises at least one of the following xanthones: alpha- and gamma-mangostins, as well as (−)-epicatechin, procyanidins A-2 and B-2, or a combination mixture thereof. More particularly, the xanthone-rich extract comprises mangostins. More particularly still, the xanthone-rich extract comprises alpha- or gamma-mangostins, or both together.

Tannins, significant constituents of mangosteen extracts, vary in terms of their biological effect, depending on their dose and method of administration. At high doses, tannins can be toxic, due their ability to interact with proteins and to chelate metals. However, tannins also are potent anti-oxidants, and this property can be exploited when they are administered at appropriate dose level. Accordingly, it important that xanthone levels, which are concentrated during the extraction and processing steps in embodiments of the invention, be separable from a directly proportional variation in the concentration of tannins. Embodiments of the inventive processes, therefore, provide for independent manipulation or modulation of the tannin concentration of the pericarp extracts, as may be appropriate for the final concentration of xanthone, and for the intended use of the composition. Such modulation provides compositions that are optimized for low cytoxicity, and high anti-oxidant properties. Demonstration of such properties is provided in the example section below, that is directed toward in vitro tests of cytotoxicity and bench studies of the oxygen radical absorbance capacity (ORAC) of compositions that are embodiments of the present invention.

The extraction methods disclosed herein result in the segregation and exclusion of a greater proportion of the tannin portion from the particular embodiment with the xanthone extract of the pericarp/rind present at about 40%. Tannin concentrations of particular embodiments where the extract is present at about 1%, do not have tannins relatively reduced in this manner, and in some embodiments, such as those with mangosteen xanthones present at 10% and about 20%, the tannin concentration may be increased by process steps, in order to increase the ORAC value (see process examples below). The cytotoxic studies presented herein below for the various extract end products obtained by these methods demonstrate a reduced cytotoxicity at various concentrations of pericarp extract in a final composition for topical use as compared to other various extraction processes that do not lower the relative tannin portion of the pericarp rind extract.

The extraction processes described herein below result in superior mangosteen pericarp extract products that can be added to other various components to formulate a composition that comprises the pericarp extract product in a final concentration of about 0.1% to about 80% of a single pharmaceutical, cosmetic, therapeutic or dermatological composition. In particular embodiments of the present invention, the extraction process results in a mixture of mangosteen pericarp extract product in a concentration amount ranging from between about 0.1% to about 80%, more particularly between about 0.3% to about 60%, and more particularly still, between about 1% to about 40% concentrate of the total weight of a composition mixture. Typically, the Garcinia mangostana (“mangosteen”) fruit rind comprises three main chemical constituents: (1) Mangostin, (2) flavonoids, and (3) tannins. The processes set forth below are useful for producing the three main chemical constituents above in different ratios and particularly useful in producing three particular embodiments of the invention: (1) a water soluble Mangosteen rind extract comprising about 1% Mangostin, (2) a Mangosteen rind extract comprising about 10% Mangostin, (3) a Mangosteen rind extract comprising about 20% Mangostin, and (4) a Mangosteen rind extract comprising about 40% Mangostin.

Accordingly, the typical extraction process according to the invention is a two-step process. The first step involves a water extraction and the second step involves an alcohol extraction. The end result is a mangosteen rind extract with a level of tannins that can be modulated either up or down, as desired, with respect to the relative presence of xanthones. Both the water and alcohol extraction steps according to the invention may include several sub-steps. Although various amounts and volumes are demarcated herein below it is understood that various other amounts, volumes, and comparable constituents may be substituted and/or added or deleted without departing from the spirit of the invention. There are two particular methods that may be practiced in conjunction with one another for obtaining the preceding three compositions or product-defined embodiments.

Two Methods for Obtaining the Three Compositions

Method A

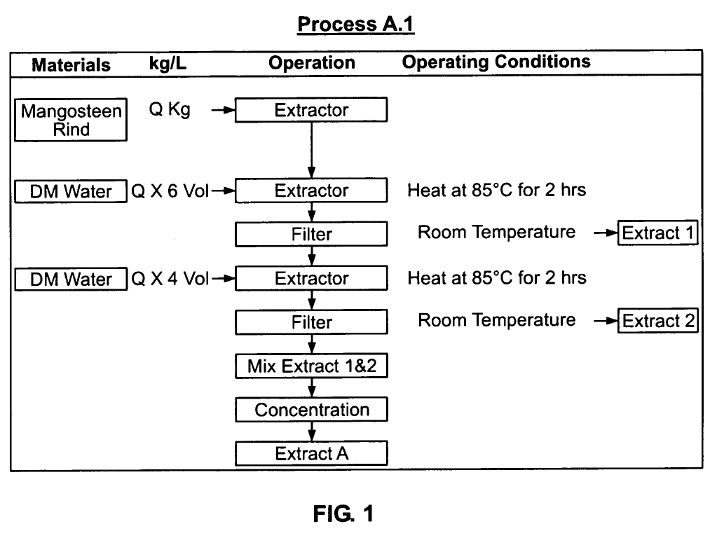

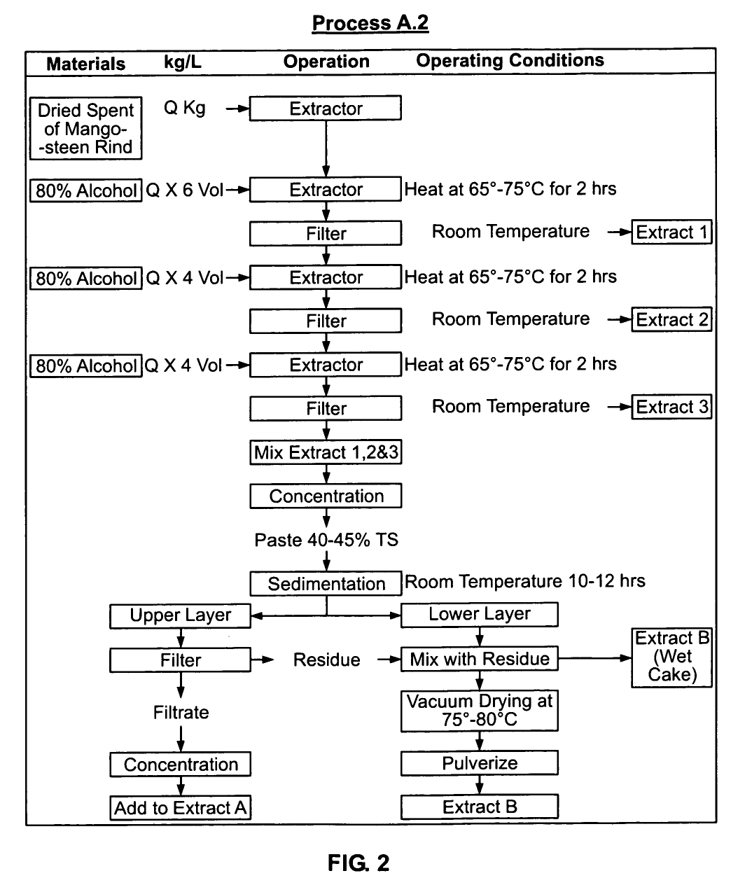

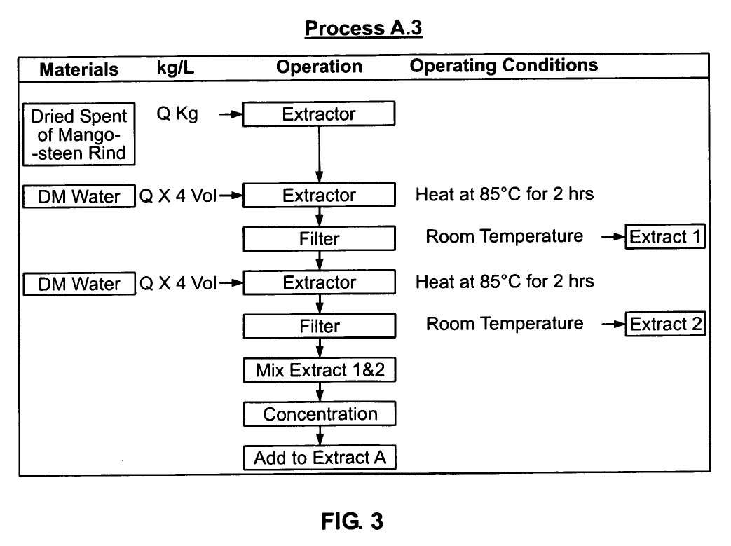

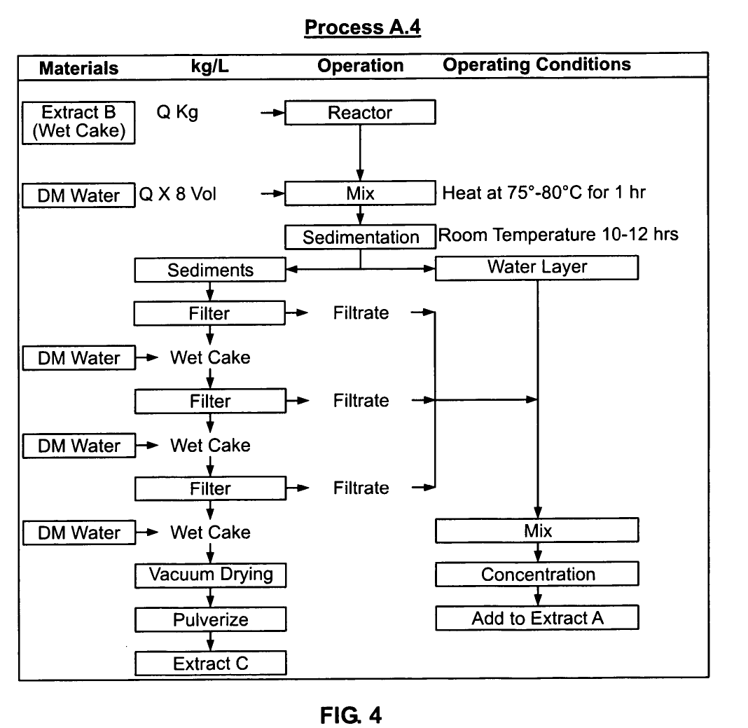

The first method (Method A) comprises three- to four separate processes each with several sub-steps. The first process (FIG. 1) involves a water extraction, the second process involves a alcohol extraction (FIG. 2), the third process involves an additional water extraction (FIG. 3), which may be followed by an additional fourth enrichment process (FIG. 4). The end result is a mangosteen rind extract enriched in mangostin. Both the water and alcohol extraction processes, according to the invention, include several sub-steps set forth below. Although various amounts and volumes are demarcated herein below it is understood that various other amounts, volumes, and comparable constituents can be substituted and/or added or deleted without departing from the spirit of the invention.

In Method A, the rind is first extracted with water and then the spent rind is subsequently extracted with organic solvent and again with water. Preferably, the first water extraction process involves the following steps. First, a known amount of dried, cleaned Mangosteen pieces are charged, in a suitable extractor, with about 6 volume of de-mineralized water (DM water). The reactor is then heated to about 85° C. by passing steam in to the outer jacket and the temperature is maintained up to about 2 hours, under circulation. After about 2 hours, the heating is stopped and the temperature is cooled to room temperature. The water extract is then filtered into a cleaned stainless steel container to give: water extract # 1.

Second, a 4 volume of DM water is charged in to the extractor. The reactor is then heated to about 85° C. by passing steam into the outer jacket and the temperature is maintained up to 2 about hours, under circulation. After about 2 hours, heating is stopped, the temperature is cooled to room temperature, and the water extract is filtered in to a cleaned stainless steel container to give: water extract # 2. Both the water extracts (1 and 2) are combined and concentrated to 20-25% of total solids. This water extract is named as Extract-A, which is a part of product # I.

The chemical composition of the product is as follows:

| Mangostin | Total Flavonoids | Total Tannins | Solubility in water |
|---|---|---|---|
| 0.1-0.3% | 5-10% | 10-20% | >95% |

In Method A, the organic solvent extraction process takes place after the water extraction process above and involves the following steps. First, a known quantity of re-dried spent of mangosteen rind from the water extraction process above is charged in to a suitable extractor with about 6 volume of 80% alcohol. The extractor is then heated to 65° C.-75° C. by passing steam into the outer jacket and the temperature is maintained at reflux condition of the extracting solvent and continued up to about 2 hours under circulation. After about 2 hours, heating is stopped and the temperature is cooled to room temperature. The alcohol extract is then filtered into a cleaned stainless steel container to give: Solvent extract # 1.

Second, 4 volume of 80% alcohol is charged into the extractor. The extractor is then heated to reflux temperature, by passing steam into the outer jacket, and reflux continued for up to about 2 hours, under circulation. After about 2 hours, heating is stopped and the temperature is cooled to room temperature. The extract is then filtered into a cleaned stainless steel container to give: Solvent extract # 2. This step may be repeated a third time to give: Solvent extract # 3. All the solvents extracts are combined and charged into a suitable reactor.

Third, the solvent from the extracts is distilled of and concentrated to a semi solid paste containing 40-45% total solids (TS). The semi solid paste is kept at room temperature for 8-10 hours for sedimentation. After sedimentation, the paste is divided into two part, an upper layer and a lower layer. The upper layer is separated and filtered through about a 5-micron filter. The lower layer and the residue after filtration of the upper layer are mixed to give a wet cake. The wet cake is then dried under constant vacuum at about 75-80° C. and pulverized to give: Extract-B. This product is a part of product # II.

Fourth, the filtered upper layer is concentrated to a paste of 20-25% of total solids. This product is highly water soluble and added in to extract-A, which is a part of product # I.

In Method A, preferably, after the organic solvent extraction process, a second water extraction process takes place for obtaining other tannins. This process involves the following steps. First, a known amount of dried spent is charged into a suitable extractor with about a 4 volume of DM water. The extractor is heated up to about 85° C. by passing steam in to the outer jacket. The temperature is maintained for up to about 2 hours under circulation. After about 2 hours, heating is stopped and the temperature is cooled to room temperature. The water extract is filtered into a cleaned stainless steel container to give: water extract # 3.

Again a 4 volume of DM water is charged in to the extractor and heated to about 85° C. by passing steam into the outer jacket. The temperature is maintained up to about 2 hours under circulation. After 2 hours, heating is stopped and the temperature is cooled to room temperature. The water extract is then filtered in to a cleaned stainless steel container to give: water extract # 4. Both the extracts (3 and 4) are combined and concentrated to 20-25% of total solids. This water extract is added into Extract-A, which is a part of product # I.

At this point the mangostin can be enriched from about 20% to >about 40%. In this process the wet cake of Extract-B, is again processed to obtain an extract containing>40% γ-mangostin, which is a part of product # III. First, a known quantity of the extract (wet cake) containing about 20% mangostin is charged in a suitable reactor with about an 8 volume of DM water and stirred well for about 30 minutes. Steam is passed in to the jacket of the reactor and the reaction mass is heated to about 75-80° C. for about 1 hour under constant stirring. The reaction mass is then cooled to room temperature by withdrawing the steam from the jacket, and unloaded into a cleaned stainless steel container and kept at room temperature for about 10-12 hours. The top layer is then decanted and the bottom sediment mass (wet cake) is collected. The sediment mass is then filtered through about a 5-micron mesh and the wet cake is collected. DM water is added to the wet cake to make it in to a slurry. Again the mass is filtered through about a 5-micron mesh to remove tannins from the product. This water washing is repeated about 2 more times. The wet cake is then dried under a vacuum at about 75-80° C. and pulverized to give: Extract-C. This product is a part of product # III. All the three water washings and the filtered top layer are combined and charged into a reactor and concentrated to a paste of 20-25% of total solids. This water extract is added into Extract-A, which is a part of product # I.

Method B

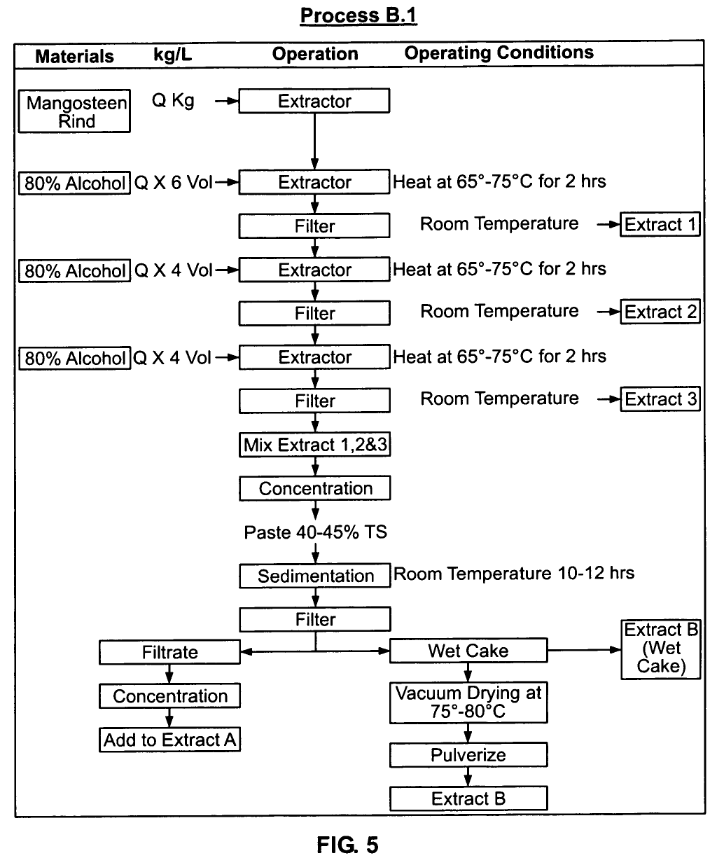

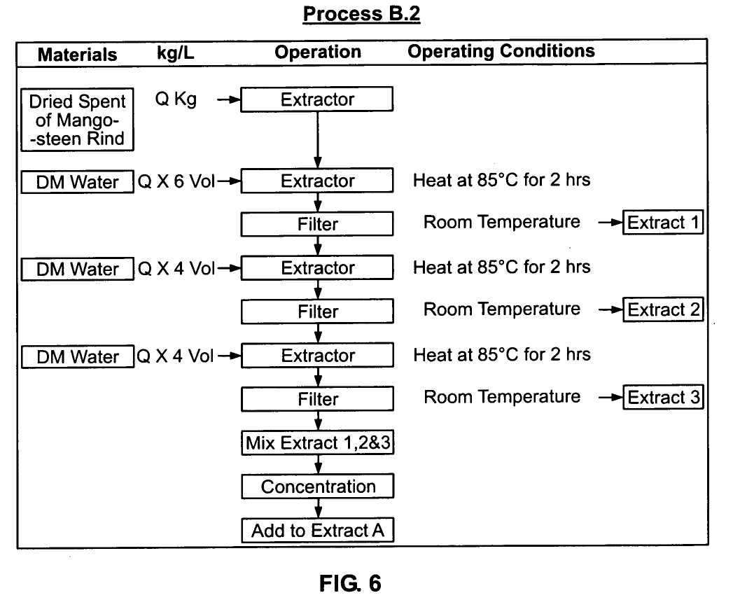

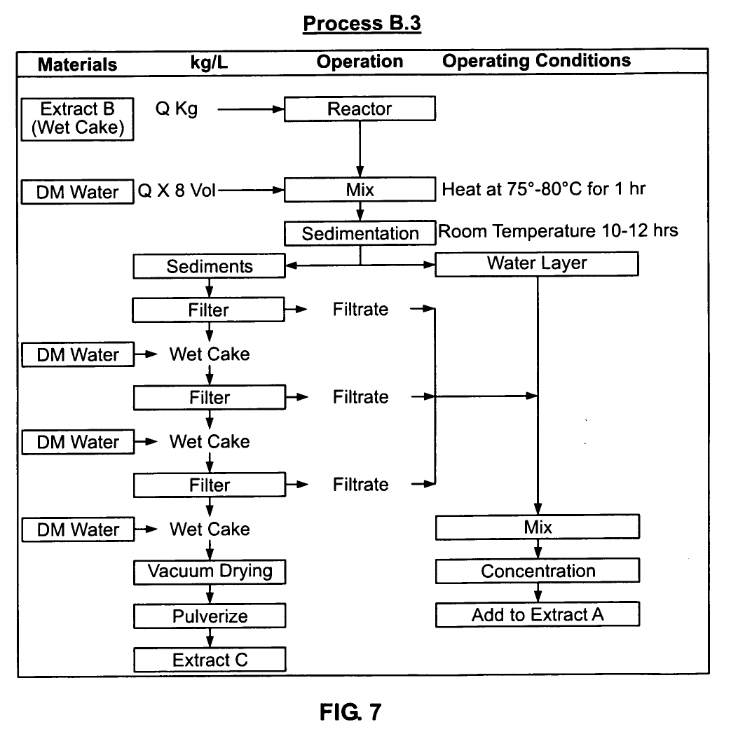

The second method (Method B) comprises three separate processes each with several sub-steps. The first process involves extraction of the rind with an organic solvent (FIG. 5), the second process involves extraction of the spent with water (FIG. 6), and the third process involves an additional enrichment process (FIG. 7). The end result is a mangosteen rind extract with a greater proportion of Mangostin. Although various amounts and volumes are demarcated herein below, it is understood that various other amounts, volumes, and comparable constituents can be substituted and/or added or deleted without departing from the spirit of the invention.

In the first process of Method B (FIG. 5) the rind is extracted first with an organic solvent and then with water. First a known quantity of dried and cleaned Mangosteen rind is charged into a suitable extractor with about a 6 volume of 80% alcohol. The reactor is then heated to about 65° C.-75° C. by passing steam into the outer jacket, the temperature is maintained at reflux condition of the extracting solvent, and is continued up to about 2 hours, under circulation. After about 2 hours, the heating is stopped and the temperature is cooled to room temperature. The alcohol extract is then filtered and stored into a cleaned stainless steel container to give: Solvent extract B# 1.

About a 4 volume of 80% alcohol is then charged into the extractor and heated by passing steam into the outer jacket. Heating is up to reflux temperature and the reflux is continued for up to about 2 hours, under circulation. After about 2 hours, heating is stopped and the temperature is cooled to room temperature. The extract is then filtered and stored into a cleaned stainless steel container to give Solvent extract B# 2. These steps are repeated once again, and the extract collected to give: Solvent extract B#3.

All the 3 solvent extracts are combined and charged it in a suitable reactor. The solvent is distilled and the extract is concentrated to a semi solid paste containing about 40-50% total solids, which is kept for about 10 hours and then filtered through a 5-micron filter. The wet cake is then dried in a vacuum at about 75-80° C. and pulverized to give: Extract-B. Both Extract-B of Method A and Extract-B of Method B are combined and this product is a part of product # II. The final product is a light brown powder, and its chemical constituent is as follows:

| Mangostin | Total Flavonoids | Total Tannins | Solubility in Alcohol |
|---|---|---|---|
| 20-25% | 40-60% | 10-15% | >95% |

The filtered upper layer is concentrated to a past of 20-25% total solids. This product is highly water soluble, and is added into extract-A, which is a part of product #1. Product #I may be added to product II to equilibrate the content of γ-mangostin, flavonoids and tannins to give the finished product II as described in the above Table. The filtrate is added into Extract-A of Method A, which is a water soluble product and is included in the product #I.

The second process of Method B (FIG. 6) involves extraction of the spent with water. First a known amount of dried spent from the first process of Method B (B.1) is charged in a suitable extractor with about 6 volume of DM water, and heated to about 85° C. by passing steam in to the outer jacket. The temperature is then maintained up to about 2 hours, under circulation. After 2 hours, heating is stopped and the temperature is cooled to room temperature. The water extract is filtered into a cleaned stainless steel container to give: water extract B#1.

About a 4 volume of DM water is then charged in to the reactor and heated up to about 85° C. by passing steam into the outer jacket. The temperature is maintained up to about 2 hours, under circulation. After 2 hours heating is stopped and the temperature is cooled to room temperature. The water extract is filtered in to a cleaned stainless steel container to give: water extract B# 2. Theses steps are repeated once more, and the extract is collected to give: water extract B#3.

All 3 water extracts are combined and charged it into a suitable reactor. The water in the extract is then distilled and the extract is concentrated to 20-25% of total solids (TS). The extract is then added to the water Extract-A of Method A, which is a part of Product #I.

The third process of Method B (FIG. 7) is for the enrichment of the extract comprising about 20% mangostin to about >40%. For this purpose the wet cake obtained during the first process of Method B (B.1) is further washed with water. A known quantity of the extract containing 20% γ-mangostin is charged in a suitable reactor with about an 8 volume of DM water, and stirred well for 30 minutes. Steam is passed into the jacket of the reactor and the reaction mass is heated to about 75-80° C. for about 1 hour under constant stirring. The reaction mass is then cooled to room temperature by withdrawing the steam from the jacket, unloaded into a cleaned stainless steel container, and kept at room temperature for about 10-12 hours. The top layer is then decanted and the bottom sediment mass is collected. The sediment mass is then filtered through a 5-micron filter and the wet cake is collected. DM water is added to the wet cake and made into a slurry, which is again filtered through a 5-micron mesh to remove tannins from the product. The water washings are repeated 2 more times. The wet cake is then dried under a vacuum at about 75-80° C. and pulverized to give: Extract-C of Method B.

Both Extract-C of Method A (A.4) and the Extract-C of Method B (B.3) are combined and this product is used for the production of product #III. The chemical composition of the product is as follows:

| Mangostin | Total Flavonoid | Total Tannins | Solubility in alcohol |
|---|---|---|---|
| 40-45% | >95% | 2-5% | >95% |

Product-#I and/or Product-#II may be added to product #III to equilibrate the content of γ-mangostin, flavonoids, and tannins to give the finished product III as described in the above Table A.

All the three water washings and the filtered top layer are then combined, charged into a reactor, and concentrated into a paste of about 20-25% of total solids. This can then be added into the Extract-A, which is a part of product # I. Extract-A (consisting of all the water-soluble portions of the different extracts is then spray dried. The parameters for spray drying are: a) inlet temperature at about 250° C., b) outlet temperature at about 108° C., and c) chamber pressure at about −5 mm in a water column. The different extracts produced from above said methods A and B are blended in such a way to obtain the three desirable products i.e., Products #I, #II and #III; such suitably standardized embodiments are outlined in the following table:

| | | | | Solubility | |
|---|---|---|---|---|---|
| | | | | in | in |
| Product | Mangostin | Total Flavonoid | Total Tannins | water | alcohol |
| I | ≧1% | ≧5% | ≧15% | >95% | — |
| II | ≧20% | ≧40% | ≧12% | — | >90% |
| III | ≧40% | ≧95% | — | — | >95% |

Methods for Obtaining an Extract Containing 40% Mangostin

To develop a Mangosteen extract containing 40% γ-Mangostin, two different methods are developed. Method 1 involves extracting the rind with DM water and then extracting the spent with an organic solvent. Method 2 involves extracting the rind directly with an organic solvent and then washing the concentrated extract with DM water.

Method 1 (for 40% Mangostin)

Method 1 comprises the following steps: After extracting the Mangosteen rind pieces with DM water (as in Method A Process # 1, above), the residual water from the spent is completely drained off. The remaining moistened Mangosteen rind pieces are known as the rind spent. A known quantity of the rind spent is then charged in to a suitable extractor with about a 6 volume of 90% organic solvent, i.e., an alcohol. The extractor is then heated to about 65° C.-75° C. by passing steam in to the outer jacket, and the temperature is maintained at reflux condition of the extracting solvent and continued up to about 2 hours. After 2 hours, heating is stopped and the temperature is cooled to room temperature. The alcohol extract is then filtered in to a well cleaned stainless steel container to give: Solvent extract # 1.

About 4 volumes of 80% alcohol is then charged into the extractor and heated to reflux temperature by passing steam in to the outer jacket. Heating and reflux are continued up to about 2 hours. After 2 hours, heating is stopped and the temperature is cooled to room temperature. The extract is then filtered in to a well cleaned stainless steel container to give: Solvent extract # 2. These steps are repeated and the extract collected to give: Solvent extract # 3. All 3 solvent extracts are combined and collect in to a suitable reactor. The solvent is distilled off from the extract and concentrated to a semi solid paste containing about 40-45% total solids (TS). The chemical composition of the concentrated extract is as follows:

| Yield | Mangostin | Total Flavonoids | Total Tannins |
|---|---|---|---|
| 10-12% | 20-25% | 40-50% | 10-15% |

The concentrated paste is then held at room temperature for about 10-12 hours for sedimentation. After sedimentation the paste is divided into upper and lower layers. Both layers are separated and analyzed, with the results that follow:

| | | | Total | |
|---|---|---|---|---|
| Layer | Yield | Mangostin | Flavonoids | Total Tannins |
| Upper | 30-40% (of TS) | 5-10% | 10-20% | 15-20% |
| Lower | 60-70% (of TS) | 25-30% | 70-80% | 5-10% |

The lower layer is separated and filtered through about a 5-micron filter. A minimum amount of water is added to make it in to a slurry and that is filtered through about a 5-micron filter and the wet cake suck dried under a constant vacuum at about 80° C. The final product is a light brown powder; the chemical composition of the final product is as follows:

| | | | | Solubility | ORAC value |
|---|---|---|---|---|---|
| | | Total | Total | in | Total hydro + |
| Yield | Mangostin | Flavonoids | Tannins | alcohol | lipo μmole TE/g |
| 7-8% | 40-45 | >95% | 2-5% | >95% | 3600-4800 |

Method 2 (for 40% Mangostin)

Method 2: A semi solid product is obtained (TDS 20-25%) from the processing of the Mangosteen rind extract containing 20% γ-mangostin, which may also be used for the enrichment of the extract containing γ-mangostin to about 40% and above. Accordingly, for the further enrichment of γ-mangostin from about 20% to about 40% and above is achieved by two different processes.

In process #1: a known quantity of the extract containing about 20% γ-mangostin is charged into a suitable reactor with about a 5 volume of DM water and stirred well for 30 minutes. A volume of about 50 L of 5% potassium hydroxide (KOH) is prepared by dissolving 2.5 kg of KOH in 50 L of DM water. This 5% KOH solution is added to the reaction mass slowly under constant stirring to bring the pH to the range of about 8.0 to about 8.2. The reaction mass is heated to about 55-60° C. for about 1 hour under constant stirring. The reaction mass is then cooled to room temperature and the pH is checked and maintain at about pH 8.0 to about 8.2 by adding 5% KOH. Again the reaction mass is heated to about 55-60° C. for about 30 minutes under constant stirring. The reaction mass is then brought down to room temperature, unloaded into a cleaned container, and kept at room temperature for about 10-12 hours. The top layer is then decanted and the bottom sediment mass is collected. The sediment mass is filtered through about a 5 micron filter and the wet cake is collected. A minimum amount of DM water is added to the wet cake to make it into slurry and again it is filtered through about a 5-micron mesh to remove excess KOH from the product. The wet cake is then dried under a vacuum at about 75° C.

| | | | | Solubility | ORAC value |
|---|---|---|---|---|---|
| | | Total | Total | in | Total hydro + |
| Yield | Mangostin | Flavonoids | Tannins | Alcohol | lipo μmole TE/g |
| 7-8% | 40-45% | >95% | 2-5% | >95% | 3600-4800 |

In process #2, a known quantity of the extract containing 20% γ-mangostin is charged in a suitable reactor with about an 8 volume of DM water and stirred well for about 30 minutes. Steam is passed in to the jacket of the reactor and the reaction mass is heated to about 75-80° C. for about 1 hour under constant stirring. The reaction mass is then cooled to room temperature by withdrawing the steam from the jacket. A brine solution is passed in to the jacket and the reaction mass is chilled to about −5° C. for about 5 hours. The temperature of the reaction mass is then brought to room temperature. The reaction mass is unloaded in a cleaned stainless steel container and kept at room temperature for about 10-12 hours. The top layer is distilled and the bottom sediment mass is collected. The sediment mass is then filtered through about a 5-micron mesh and the wet cake is collected. DM water is added to the wet cake to make it in to a slurry. Again it is filtered through about 5-micron mesh to remove tannins from the product. The water washings is then repeated about 2 more times, the wet cake is then dried under a vacuum at about 75° C., and pulverized. The chemical composition of the extract is as follows:

| | | | | Solubility | ORAC value |
|---|---|---|---|---|---|
| | | Total | Total | in | Total hydro + |
| Yield | Mangostin | Flavonoid | Tannins | alcohol | lipo μmole TE/g |
| 7-8% | 40-45 | >95% | 2-5% | >95% | 3600-4800 |

All the three water washings are then combined, charged into a reactor, concentrated in to a paste of 20-25% TS, and the product is spray dried. The parameters for spray drying are: (a) inlet temperature: about 250° C., (b) outlet temperature: about 108° C., and (c) chamber pressure: about −5. (mm in water column).

This water product is water soluble and may be used as a diluent to the Mangosteen rind extracts to prepare formulations comprising a desirable quantity of γ-mangostin. The final product is a brown powder. This product is referred to as “Product (a), and the same is added into above mentioned product I in desirable quantity. The chemical composition of the product is as follows:

| | | Total | Total | Solubility in |
|---|---|---|---|---|
| Yield | Mangostin | Flavonoids | Tannins | water |
| 7-10% | 0.1-0.3% | 5-10% | 10-20% | >95% |

### Examples of Production Processes and In Vitro and Bench-Level Studies

### Process Examples

Process A.1: Extraction of Mangosteen Rind with Water

Method-A: In method-A, the rind is first extracted with water and then the spent is subsequently extracted with organic solvent and again with water, as depicted in FIG. 1, and as follows:
1. Charging a known amount of dried, cleaned Mangosteen rind pieces in a suitable extractor.
2. Charging 6 volume of de-mineralized water (DM water).
3. Starting the heating of the extractor by passing the steam in to the outer jacket.
4. Heating to 85° C. and maintain the temperature up to 2 hours, under circulation.
5. Ceasing heat input, after 2 hours, and cooling to room temperature.
6. Filtering the water extract into a cleaned stainless steel container. (water extract # 1)
7. Charging 4 volume of DM water into the extractor.
8. Starting the heating of the reactor by passing the steam into the outer jacket.
9. Heating to 85° C. and maintain the temperature up to 2 hours, under circulation.
10. Ceasing heat input after 2 hours, and cooling to room temperature.
11. Filtering the water extract into a cleaned stainless steel container (water extract # 2)

12. Combining both the water extracts and concentrate to 20-25% of total solids. This water extract is named as Extract-A, which is a part of product # I. The chemical composition of the product is as follows:

| | Total | | |
|---|---|---|---|
| Mangostin | Flavonoids | Total tannins | Solubility in water |
| 01.-0.3% | 5-10% | 10-20% | >95% |

Process A.2: Extraction of Mangosteen Spent (After Water Extraction) with Organic Solvent

Process A.2, as depicted in FIG. 2, operates as follows:
1. Charging known quantity of re-dried spent of mangosteen rind from process A. 1, in to a suitable extractor.
2. Charging 6 volume of 80% alcohol, i.e., an alcohol.
3. Initiating the heating of the reactor by passing the steam into the outer jacket.
4. Heating up to 65° C.-75° C. and maintaining the temperature at reflux condition of the extracting solvent and continue up to 2 hours, under circulation.
5. Ceasing the heating after 2 hours, and cooling to room temperature.
6. Filtering the alcohol extract into a cleaned stainless steel container (Solvent extract # 1).
7. Charging 4 volume of 80% alcohol into the reactor
8. Initiating the heating of the reactor by passing the steam into the outer jacket.
9. Heating up to reflux temperature and continuing the reflux up to 2 hours, under circulation.
10. Ceasing the heat input after 2 hours, cooling to room temperature.
11. Filtering the extract into a cleaned stainless steel container (Solvent extract # 2).
12. Repeating the steps 7 to 11 and collecting the extract (Solvent extract # 3).
13. Combining all the 3 solvents extracts and charge into a suitable reactor.
14. Distilling off the solvent from the extract and concentrate to a semi solid paste containing 40-45% total solids.
15. Holding the semi solid paste in room temperature for 8-10 hours for sedimentation.
16. Dividing the paste, after sedimentation, into two parts, an upper layer and a lower layer
17. Separating the lower layer and filter through 5-micron filter.
18. Mixing the lower layer and the residue after filtration of upper layer to give wet cake.
19. Drying the wet cake under constant vacuum at 75-80° C. and pulverize it (Extract-B). (This product is a part of product # II.)
20. Concentrating the filtered upper layer to a paste of 20-25% of total solids. (This product is highly water soluble and added in to extract-A, which is a part of product #I.)
   Process A.3: Extraction of spent with water

In this process, as depicted in FIG. 3, the spent after solvent extraction is once again extracted with water to obtain other tannins.
1. Charging known amount of dried spent into a suitable extractor.
2. Charging 4 volume of DM water.
3. Initiating the heating of the reactor by passing the steam in to the outer jacket.
4. Heating up to 85° C. and maintain the temperature up to 2 hours under circulation.
5. Ceasing heat input after 2 hours, cooling to room temperature.
6. Filtering the water extract into a cleaned stainless steel container. (water extract # 1)
7. Charging 4 volume of DM water in to the reactor
8. Initiating the heating of the reactor by passing the steam into the outer jacket.
9. Heating to 85° C. and maintain the temperature up to 2 hours under circulation.
10. Ceasing heat input after 2 hours, and cooling to room temperature.
11. Filtering the water extract in to a cleaned stainless steel container. (water extract # 2)
12. Combining both the extracts and concentrate to 20-25% of total solids. (This water extract is added into Extract-A of process A.1, which is a part of product # I.)
   Process A.4: Enrichment of Mangostin from 20% to >40%.

In this process (see FIG. 4) the wet cake of Extract-B, of process A.2, is again processed to obtain an extract containing >40% γ-mangostin, which is a part of product # III.
1. Charging a known quantity of the extract (wet cake) containing 20% γ-mangostin in a suitable reactor.
2. Adding 8 volume of DM water in to the reactor.
3. Stirring well for 30 minutes
4. Passing steam into jacket of the reactor and heat the reaction mass to 75-80° C. for 1 hour under constant stirring.
5. Cooling the reaction mass to room temperature by withdrawing the steam from the jacket.
6. Unloading the reaction mass in a cleaned stainless steel container
7. Holding the reaction mass in room temperature for 10-12 hours.
8. Decanting the top layer and collect the bottom sediment mass.
9. Filtering the sediment mass through 5-micron mesh and collect the wet cake.
10. Adding DM water to the wet cake and make it in to slurry.
11. Filtering through 5-micron mesh to remove tannins from the product.
12. Repeating the water washings twice more.
13. Drying the wet cake under vacuum at 75-80° C. and pulverize it (Extract-C); this product is a part of product # III.
14. Combining all the three water washings and the filtered top layer, and charging into a reactor.
15. Concentrating the material to a paste of 20-25% of total solids; this water extract is added into Extract-A of process A. 1, which is a part of product # I.)
   Process B.1: Extraction of the Rind with Organic Solvent

Method B: In method-B, as depicted in FIG. 5) the rind is extracted first with organic solvent and then with water.
1. Charging known quantity of dried and cleaned Mangosteen rind in to a suitable extractor.
2. Charging 6 volume of 80% alcohol.
3. Initiating the heating of the reactor by passing the steam into the outer jacket.
4. Heating up to 65° C.-75° C. and maintain the temperature at reflux condition of the extracting solvent and continue up to 2 hours, under circulation.
5. Ceasing heat input after 2 hours, and cooling to room temperature.
6. Filtering the alcohol extract into a cleaned stainless steel container. (solvent extract # 1)
7. Charging 4 volume of alcohol into the reactor
8. Initiating the heating of the reactor by passing the steam into the outer jacket.
9. Heating up to reflux temperature and continue the reflux up to 2 hours, under circulation.
10. Ceasing heat input after 2 hours, and cooling to room temperature.
11. Filtering the extract into a cleaned stainless steel container. (solvent extract # 2)
12. Repeating the steps 7 to 11 and collect the extract (solvent extract # 3).
13. Combining all the 3 solvents extracts and charge it in a suitable reactor.
14. Distilling off the solvent from the extract and concentrate to a semi solid paste containing 40-45% total solids.
15. Holding the paste for about 10 hours.
16. Filtering the paste through 5-micron filter.

17. Drying the wet cake in vacuum at 75-80° C. and pulverize it (Extract-B). Both extract-B of process # A.2 and extract-B of process # B.1 are combined and this product is a part of product # II. (Product-#I may be added to product #II to equilibrate the content of γ-mangostin, flavonoids and tannins to give the finished product 11 as described in Table 1A). The final product is a light brown powder and its chemical composition is as follows:

| | Total | | |
|---|---|---|---|
| γ-mangostin | Flavonoids | Total Tannins | Solubility in alcohol |
| 20-25% | 40-60% | 10-15% | >90% |

18. The filtrate is added into Extract-A of process A.1, which is a water soluble product and is included in the product-I.
   Process B.2: Extraction of the Spent with Water

Process B.2 is depicted in FIG. 6, as operates as follows:
1. Charging a known amount of dried spent from process B.1 in a suitable extractor.
2. Charging 6 volume of DM water.
3. Initiating the heating of the extractor by passing the steam in to the outer jacket.
4. Heating to 85° C. and maintaining the temperature up to 2 hours, under circulation.
5. Ceasing heat input after 2 hours, cooling to room temperature.
6. Filtering the water extract into a cleaned stainless steel container. (water extract # 1)
7. Charging 4 volume of DM water into the extractor
8. Initiating the heating of the extractor by passing the steam into the outer jacket.
9. Heating to 85° C. and maintain the temperature up to 2 hours, under circulation.
10. Ceasing heat input after 2 hours, cooling to room temperature.
11. Filtering the water extract in to a cleaned stainless steel container. (water extract # 2)
12. Repeating the steps 7 to 11 and collect the extract (water extract # 3).
13. Combining all the 3 water extracts and charge it into a suitable reactor.
14. Distilling off the water in the extract and concentrate to 20-25% of total solids (TS).
15. Adding this extract to the water extract-A of process A.1, which is a part of Product #I.
   Process B.3: Enrichment of Extract Containing 20% γ-mangostin to >40%

In process B.3 (FIG. 7), the wet cake obtained during the process # B.1 is further washed with water.
1. Charging a known quantity of the extract containing 20% γ-mangostin in a suitable reactor.
2. Adding 8 volume of DM water in to the reactor.
3. Stirring well for 30 minutes
4. Passing steam into jacket of the reactor and heating the reaction mass to 75-80° C. for 1 hour under constant stirring.
5. Cooling the reaction mass to room temperature by withdrawing the steam from the jacket.
6. Unloading the reaction mass in a cleaned stainless steel container
7. Holding the reaction mass in room temperature for 10-12 hours.
8. Decanting the top layer and collect the bottom sediment mass.
9. Filtering the sediment mass through 5-micron filter and collect the wet cake.
10. Adding DM water to the wet cake and make it into slurry.
11. Filtering through 5-micron mesh to remove tannins from the product.
12. Repeating the water washings 2 more times.

13. Drying the wet cake under vacuum at 75-80° C. and pulverize it (Extract-C). Both extract-C of process A.4 and the extract-C of process B.3 are combined and this product is used for the production of product #III. (Product-I and/or Product-II may be added to product III to equilibrate the content of γ-mangostin, flavonoids and tannins to give the finished product III as described in Table 1A.) The chemical composition of the product is as follows:

| | Total | | |
|---|---|---|---|
| Mangostin | Flavonoid | Total Tannins | Solubility in alcohol |
| 40-45% | >95% | 2-5% | >95% |

14. Combining all the three water washings and the filtered top layer, and charging into a reactor.
15. Concentrating the combined washings to a paste of 20-25% of total solids.

This is added into the Extract-A, which a part of product # I. The extract-A (consisting of all the water-soluble portions of different extracts) is spray dried. The parameters for spray drying are: a) inlet temperature:250° C., b) outlet temperature:108° C. and c) chamber pressure: −5 (mm of water column)

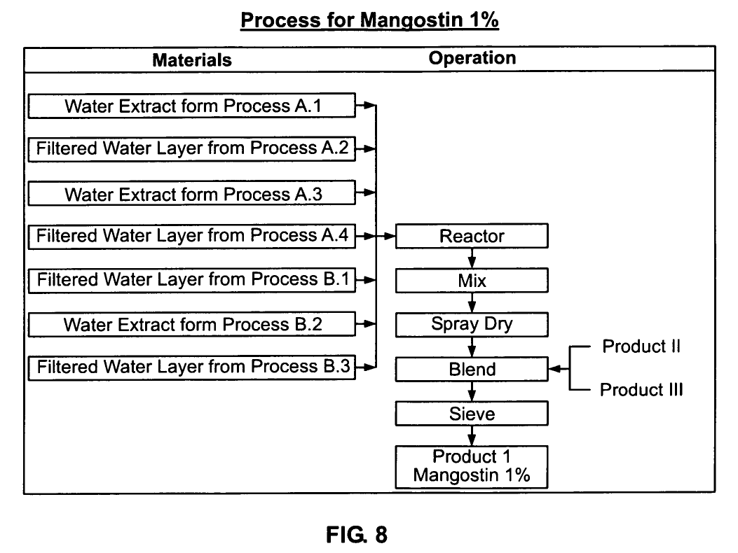

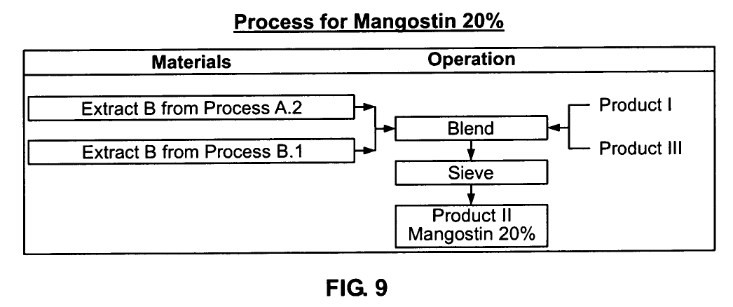

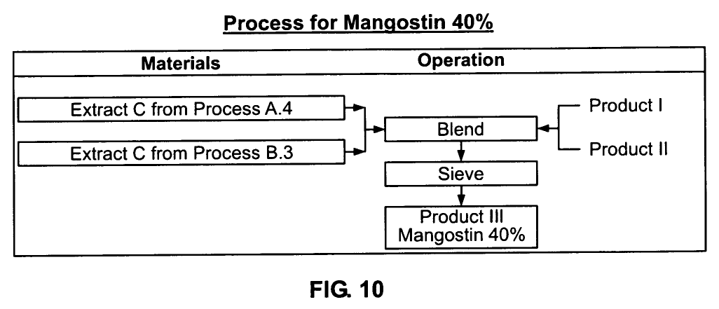

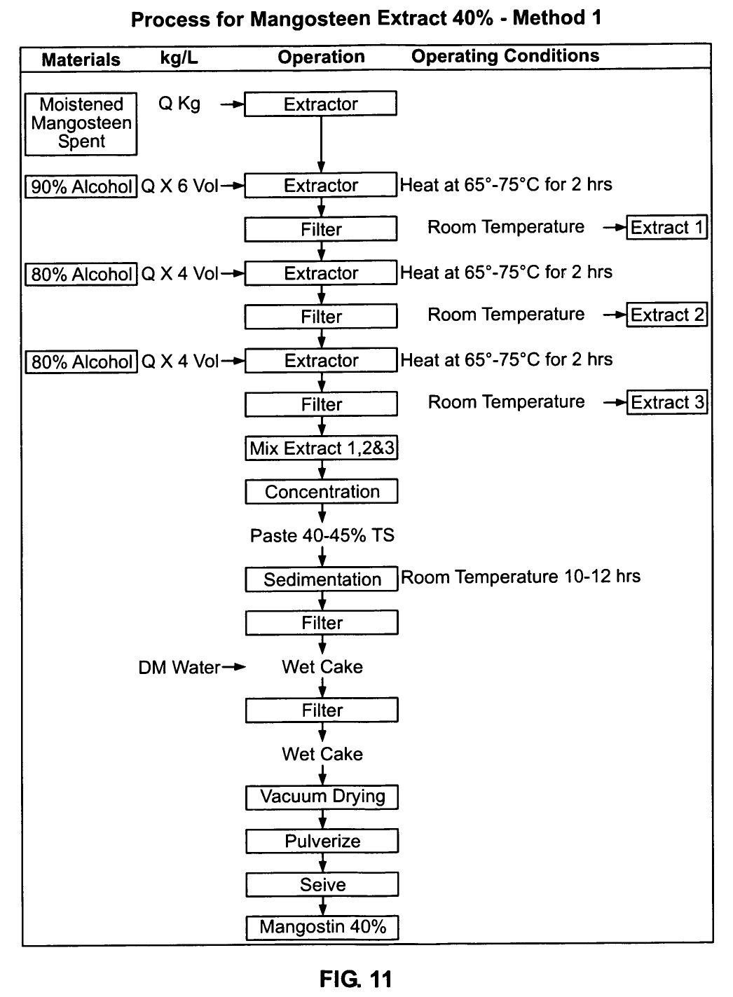

The different extracts produced from above said methods A and B are blended in such a way to obtain the three desirable products i.e., Product # I, II and III. An overview of the process for Product I, 1% mangostin, is provided in FIG. 8; and overview of the process for 20% Mangostin is provided in FIG. 9. An overview of the process for 40% Mangostin is provided in FIG. 10; details of the 40% Mangostin process options are provided in FIGS. 11 -13. The chemical and physical parameters of these three embodiments, identified here as Products I, II, and III, are shown in the following table:

| | | | | Solubility | |
|---|---|---|---|---|---|
| | | Total | | | in |
| Product # | Mangostin | Flavonoid | Total Tannins | In water | alcohol |
| I | ≧1% | ≧5% | ≧15% | ≧95% | — |
| II | ≧20% | ≧40% | ≧12% | — | ≧90% |
| III | ≧40% | ≧95% | — | — | ≧95% |

Process for Mangosteen Extract Containing 40% γ-mangostin

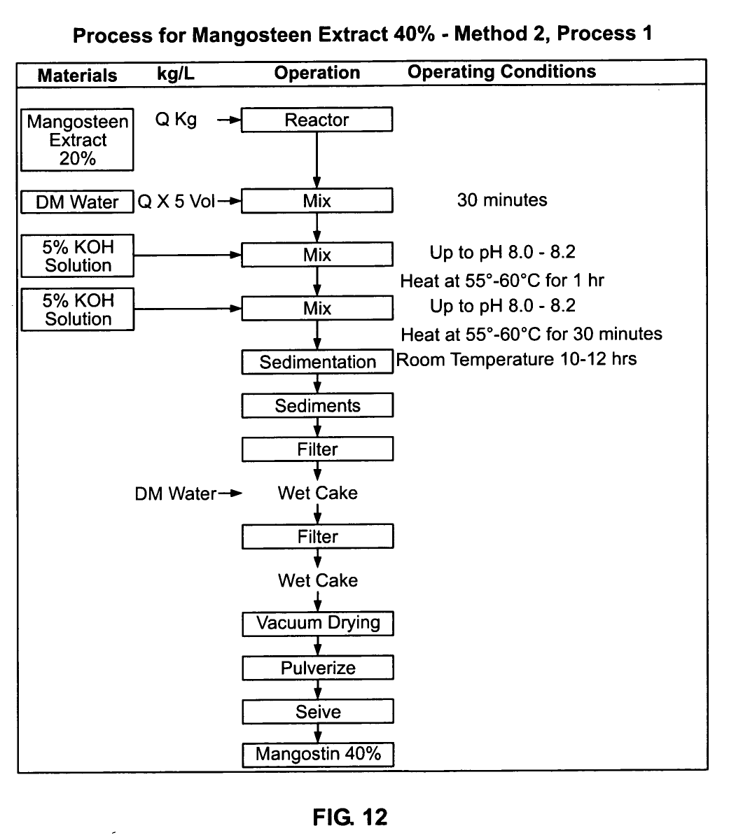

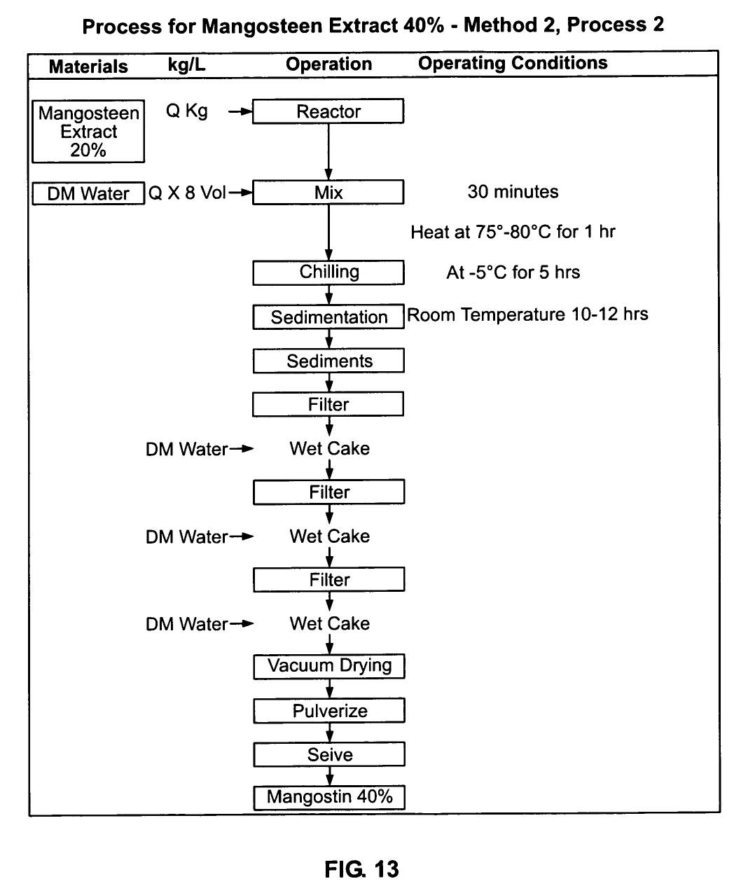

To develop a Mangosteen extract containing 40% Mangostin, two different methods are provided. Method 1 (FIG. 11) entails extracting the rind with DM water, and then extracting the spent with organic solvent. Method 2 entails extracting the rind directly with organic solvent (FIG. 12) and then washing the concentrated extract with DM water (FIG. 13).

Method 1 for 40% Mangostin (see FIG. 11)

1. After extracting the Mangosteen rind pieces with DM water (Ref: Process # 1), the residual water from the spent is completely drained off. The remaining moistened Mangosteen rind pieces is known as Mangosteen spent.
2. Charging known quantity of Mangosteen spent in to a suitable extractor.
3. Charging 6 volume of 90% organic solvent, i.e., an alcohol.
4. Initiating the heating if the reactor by passing the steam into the outer jacket.
5. Heating to 65° C.-75° C. and maintain temperature at reflux condition of the extracting solvent and continuing up to 2 hours.
6. Ceasing heat input after 2 hours, and cooling to room temperature.
7. Filtering the alcohol extract in to a well cleaned stainless steel container. (solvent extract # 1)
8. Charging 4 volume of 80% alcohol into the extractor
9. Initiating the heating of the reactor by passing the steam in to the outer jacket.
10. Heating up to reflux temperature and continuing the reflux up to 2 hours.
11. Ceasing the heating after 2 hours, and cooling to room temperature.
12. Filtering the extract in to a well cleaned stainless steel container. (solvent extract # 2)
13. Repeating the steps 7 to 11 and collect the extract (solvent extract # 3).
14. Combining all the 3 solvent extracts and collect it in a suitable reactor.

15. Distilling off the solvent from the extract and concentrate to a semi solid paste containing 40-45% total solids (TS). The chemical composition of the concentrated extract is as follows:

| Yield | Mangostin | Total Flavonoids | Total Tannins |
|---|---|---|---|
| 10-12% | 20-25% | 40-50% | 10-15% |

16. Holding concentrated paste in room temperature for 10-12 hour for sedimentation. After sedimentation the paste is divided into upper and lower layers. Both layers are separated and analyzed.

| | | | Total | |
|---|---|---|---|---|
| Layer | Yield | Mangostin | Flavonoids | Total Tannins |
| Upper | 30-40% (of TS) | 5-10% | 10-20% | 15-20% |
| Lower | 60-70% (of TS) | 25-30% | 70-80% | 5-10% |

17. Separating the lower layer and filter through 5-micron filter.
18. Adding the minimum amount of water and make it in to a slurry,
19. Filtering through 5-micron filter and suck dry the wet cake.

20. Drying the wet cake under constant vacuum at 80° C. The Final product is a light brown powder, with a chemical composition as follows:

| | | | | Solubility | ORAC value |
|---|---|---|---|---|---|
| | | Total | Total | in | Total hydro + |
| Yield | Mangostin | Flavonoids | Tannins | alcohol | lipo μmole TE/g |
| 7-8% | 40-45 | >95% | 2-5% | >95% | 3600-4800 |

Method 2 for 40% Mangostin:

The semi solid product obtained (TDS 20-25%) from the Process for the Mangosteen containing 20% γ-mangostin may also be used for the enrichment of the extract containing γ-mangostin to 40% and above. For the further enrichment of γ-mangostin from about 20% to 40% and above is achieved by two different processes.

Process # 1 of Method 2 for 40% Mangostin (see FIG. 12)

1. Charging a known quantity of the extract containing ≅20% γ-mangostin into a suitable reactor.
2. Adding 5 volume of DM water into the reactor.
3. Stirring well for 30 minutes
4. Preparing 50 L of 5% potassium hydroxide (KOH) by dissolving 2.5 kg of KOH in 50 L of DM water.
5. Adding this 5% KOH solution to the reaction mass slowly under constant stirring to bring the pH to 8.0 to 8.2.
6. Heating the reaction mass to 55-60° C. for 1 hour under constant stirring.
7. Cooling the reaction mass to room temperature.
8. Checking the pH and maintain the pH 8.0 to 8.2 by adding 5% KOH.
9. Heating the reaction mass to 55-60° C. for 0.5 hour under constant stirring.
10. Cooling the reaction mass to room temperature.
11. Unloading the reaction mass in a cleaned container
12. Keeping the reaction mass in room temperature for 10-12 hours.
13. Decanting the top layer and collect the bottom sediment mass.
14. Filtering the sediment mass through 5 micron filter and collect the wet cake.
15. Adding minimum amount of DM water to the wet cake and make it in to slurry.
16. Filtering through 5-micron mesh to remove excess KOH from the product.

17. Drying the wet cake under vacuum at 75° C.

| | | | | Solubility | ORAC value |
|---|---|---|---|---|---|
| | | Total | Total | in | Total hydro + |
| Yield | Mangostin | Flavonoids | Tannins | alcohol | lipo μmole TE/g |
| 7-8% | 40-45% | >95% | 2-5% | >95% | 3600-4800 |

Process # 2 of Method 2 for 40% Mangostin (see FIG. 13)
1. Charging known quantity of the extract containing 20% γ-mangostin in a suitable reactor.
2. Adding 8 volume of DM water in to the reactor.
3. Stirring well for 30 minutes
4. Passing steam into jacket of the reactor and heat the reaction mass to 75-80° C. for 1 hour under constant stirring.
5. Cooling the reaction mass to room temperature by withdrawing the steam from the jacket.
6. Passing a brine solution in to the jacket and chill the reaction mass to −5° C. for 5 hour.
7. Allowing the reaction mass to return to room temperature.
8. Unloading the reaction mass in a cleaned stainless steel container
9. Holding the reaction mass in room temperature for 10-12 hours.
10. Decanting the top layer and collect the bottom sediment mass.
11. Filtering the sediment mass through 5-micron mesh and collect the wet cake.
12. Adding DM water to the wet cake and make it into slurry.
13. Filtering through 5-micron mesh to remove tannins from the product.
14. Repeating the water washings 2 more times.

15. Drying the wet cake under vacuum at 75° C. and pulverize it. The chemical composition of the extract is as follows:

| | | | | Solubility | ORAC value |
|---|---|---|---|---|---|
| | | Total | Total | in | Total hydro + |
| Yield | Mangostin | Flavonoid | Tannins | alcohol | lipo μmole TE/g |
| 7-8% | 40-45% | >95% | 2-5% | >95% | 3600-4800 |

16. Combining all the three water washings the filtered top layer, and charging into a reactor.
17. Concentrating the it to a paste of 20-25% TS
18. Spray-drying the product.

This water product is water soluble and used as diluent to the Garcinia rind extracts to prepare formulations containing desirable quantity of γ-mangostin. Spray dry the extract and the parameters for spray drying are: (a) inlet temperature: 250° C., (b) outlet temperature: 108° C. and (c) chamber pressure: −5

In Vitro and Bench Level Studies

The safety and effectiveness of the mangosteen compositions described herein is demonstrated by the following examples, which are listed for only illustrative purposes, and are not limiting instances of prophylactic or therapeutic use. A therapeutic composition of the mangosteen pericarp mixture (Xanomax® 10%) was prepared according to the embodiments described herein and used as a test article in in vitro cytoxicity tests of safety and bench level studies of anti-oxidant capacity. Additionally, examples of various product forms of the mangosteen compositions are described, and examples of their beneficial effects on human health are provided.

Toxicity Study 1

The purpose of this study was to evaluate an extract of mangosteen for its level of cytotoxicity in a mammalian system. Accordingly, a serially diluted extract of a 10% mangosteen pericarp test article for cytotoxicity to mammalian cells in culture (L-929 mouse fibroblast cells, from the American Type Culture Collection: CCL-1). The test article, originally a 10% mangosteen preparation) was incubated in an appropriate volume of cell culture medium (Earle's Minimal Essential Medium, E-MEM) in a sterile vessel for 24 to 25 hours at 37 ±1° C. At the end of the extraction period, the extract was decanted from the test article. A series of eight (8) dilutions of the extract was prepared in culture medium in a two-fold dilution sequence. After the dilutions were prepared, the maintenance culture medium was removed from test culture wells and replaced with 1 ml of article test dilutions. The cytotoxic positive control medium (100 μmole CdCl₂), test article extract dilutions, and control extracts were added at the same time to the culture plate in triplicate wells. The cell culture plates were incubated for 72 hours at 37 ±1° C. in a humidified atmosphere of 5±1% CO2 in air. The cultures were evaluated for cytotoxic effects by microscopic examination at 24, 48, and 72±4 hours of incubation, and the results scored and recorded.

Final evaluation of the validity of the assay and test article results was based upon the criteria listed below and scientific judgment. When scoring lysis, each culture was rated relative to the amount of lysis displayed by the negative control. The negative control was considered a “0” if cells display at least 70% viability. Less than 70% cell viability and/or greater than 10% degeneration displayed in negative control dishes was considered an indication of a problem inherent to the cells or the culturing technique, and the test results therefore not used in any analysis. Only valid assays, with appropriate positive and negative control results were included in the analysis. Positive controls display a moderate to strong cytotoxic reaction, resulting in a score of “3” or “4”. Negative controls display no cytotoxic reaction.

The test article was submitted as a powder and maintained at room temperature. A stock solution was prepared by mixing 4.2 g of the test article with 21 mL of E-MEM+5% fetal bovine serum (FBS). The mixture was incubated at 37±1° C. for 24-25 hours. After incubation, the mixture had separated into two layers. A layer visually appearing free of particulate was removed from the tube and transferred to a centrifuge tube. The liquid was then centrifuged at 3000 rpm for 10 minutes. The resultant supernatant was then used for testing. Lower doses were prepared by dilution in E-MEM +5% FBS from this stock immediately prior to use. An aliquot of the stock solution was taken for pH measurement.

Criteria for evaluating cytotoxicity included morphologic changes in cells, such as granulation, crenation, or rounding, and loss of viable cells from the monolayer by lysis or detachment. The validity of the test requires that negative control cultures maintain a healthy normal appearance throughout the duration of the test. Degrees of toxicity are scored according to the scheme in Table 2:

Table 2: Cytotoxicity Scoring Criteria

TABLE 2
Cytotoxicity Scoring Criteria

| Grade | Reactivity | Conditions of Cultures |
|---|---|---|
| 0 | None | Discrete intracytoplasmic granules; no cell lysis |
| 1 | Slight | Not more than 20% of the cells are round, loosely |
| | | attached, and without intracytoplasmic granules; |
| | | occasional lysed cells are present |
| 2 | Mild | Not more than 50% of the calls are round and |
| | | devoid of intracytoplasmic granules; no extensive |
| | | cell lysis and empty areas between cells |
| 3 | Moderate | Not more than 70% of the cell layers contain |
| | | rounded cells and/or are lysed |
| 4 | Severe | Nearly complete destruction of the cell layers |

After the initial incubation of the stock mixture, the medium was found to be a dark brown color, it contained high levels of flocculent powder, and the pH of the mixture was 4.03. During the initial two scoring periods, the top two doses could not be scored due to the level of debris in order to visualize the cells during the final scoring period, the neat dosed wells were washed with phosphate buffered saline. This allowed visualization of the monolayer and subsequent scoring. As shown in Table 3, two dose levels (dilutions of 1:4 and 1:8) were noted to have mildly affected the cells. However, at the 1:16 dilution, the cells appeared morphologically abnormal, consistent with complete cell death.

Table 3: Cyotoxicity Data, from Study 1

TABLE 3
Cyotoxicity Data, from Study 1

| Test Articles and Controls | Cytotoxicity Scores at 72 Hr |
|---|---|
| Test Article - Neat | 0/0/0 |
| Test Article - 1:2 | 0/010 |
| Test Article - 1:4 | 2/2/2 |
| Test Article - 1:8 | 2/212 |
| Test Article - 1:16 | 4/4/4 |
| Test Article - 1:32 | 1/1/1 |
| Test Article - 1:64 | 0/0/0 |
| Test Article - 1:128 | 0/0/0 |
| Test Article - 1:256 | 0/0/0 |
| Positive Control | 4/4/4 |
| Intermediate Control | 2/2/2 |
| Negative Control | 0/0/0 |
| Cell Control | 0/0/0 |

The test article, Xanomax® 10%, scored “4” at 72±4 hours and is considered toxic at that level under the conditions of this test. However, the four more concentrated dose levels did not induce the types of morphological changes associated with significant toxicity. At the 1:32 dose, the test article did not induce substantial morphological changes. Thus, the test article would be considered to have passed the assay at the 1:32 dilution. At higher levels of dilution, no evidence of cytotoxicity as apparent.

Toxicity Study 2

The purpose of this study was to evaluate a serially diluted extract of a 40% mangosteen pericarp test article for cytotoxicity to mammalian cells in culture. The test article was incubated in an appropriate volume of E-MEM in a sterile vessel for 24 to 25 hours at 37 f 1° C. After measures were taken to create a clear supernatant, a series of eight (8) dilutions of the extract were prepared in E-MEM in a two-fold dilution sequence. After the dilutions were prepared, the maintenance culture media was removed from test culture wells and replaced with 1 ml of test media dilutions in triplicate wells. The positive control media, test article extract, and control extracts were added at the same time to the culture plate in triplicate wells. The plates were incubated for 72 hours at 37±1° C. in a humidified atmosphere of 5±1% CO₂ in air. The cultures were evaluated for cytotoxic effects by microscopic examination at 24,48 and 72±4 hours of incubation and the results scored and recorded. After the 24 hour extraction the liquid was transferred to a 50 mL conical tube and centrifuged for 10 minutes at 3000 rpm. A small pellet of powder created by the separation was discarded, the resultant supernatant was clear with a light brown color and a pH of 7.5.

Criteria for evaluating cytotoxicity included morphologic changes in cells, such as granulation, crenation, or rounding, and loss of viable cells from the monolayer by lysis or detachment. The validity of the test requires that negative control cultures maintain a healthy normal appearance throughout the duration of the test. Degrees of toxicity are scored as in Table 1, above:

The assay results are presented in Table 3. Upon microscopic evaluation, a substantial amount of test article debris was noted in all wells treated with the neat extract. Final evaluation on day three was performed by two scientists. While it appeared that the cells treated with the neat extract were more rounded and showed some crenation as compared to the negative control, they were alive and extensive lysis was not noted. Although the cells in wells treated with the 1:4 dilution did appear dead (completely rounded and crenated), the cells appeared attached.

Table 4: Cytoxicity Data, from Study 2

TABLE 4
Cytotoxicity Data

| Test Article, | Sample Extract | | Cytoxicity Scores | | |
|---|---|---|---|---|---|
| Dilutions, and Controls | Size | (mL) | 24 Hr | 48 Hr | 72 Hr |
| Xanomax ® 40% | 4.0 g | 20.0 | 4/4/4 | 4/4/4 | 2/2/2 |
| Test Article - 1:2 | n/a | n/a | 2/2/2 | 2/2/2 | 2/2/2 |
| Test Article - 1:4 | n/a | n/a | 4/4/4 | 4/4/4 | 4/4/4 |
| Test Article - 1:8 | n/a | n/a | 0/0/0 | 0/0/0 | 0/0/0 |
| Test Article - 1:16 | n/a | n/a | 0/0/0 | 0/0/0 | 0/0/0 |
| Test Article - 1:32 | n/a | n/a | 0/0/0 | 0/0/0 | 0/0/0 |
| Test Article - 1:64 | n/a | n/a | 0/0/0 | 0/0/0 | 0/0/0 |
| Test Article - 1:128 | n/a | n/a | 0/0/0 | 0/0/0 | 0/0/0 |
| Test Article - 1:256 | n/a | n/a | 0/0/0 | 0/0/0 | 0/0/0 |
| Positive Control | 0.1 mL | 9.9 | 4/4/4 | 4/4/4 | 4/4/4 |
| Intermediate Control | 1.9 g | 9.5 | 2/2/2 | 2/2/2 | 2/2/2 |
| Negative Control | 1.9 g | 9.5 | 0/0/0 | 0/0/0 | 0/0/0 |
| Cell Control | NA | 10.0 | 0/0/0 | 0/0/0 | 0/0/0 |

The test article, Xanomax® (40%), scored “4” at 24 and 48±4 hours and “2” at 72±4 hours at the neat concentration and is considered non-toxic under the conditions of this test at this dose level. At the 1:2 dilution, test article scored “2” at the 24, 48 hour and 72 hour observation period, while at the 1:4 dose level, it induced a score of “4” at the 24, 48 and 72 hour observation periods. Thus, the 1:2 dose level would be considered to have induced a non-toxic response but the 1:4 dose was found to be toxic. All other dose levels scored “0” and are considered non-toxic under the conditions of this study.

ORAC and COX Analyses

Oxygen radical absorbance capacity (ORAC) analysis is a reliable and recognized standard test of the antioxidant capacity of natural products. More specifically, this assay provides a measure of the scavenging capacity of antioxidants against the peroxyl radical, which is one of the most common reactive oxygen species (ROS) found in the body. ORAC/hydro reflects water-soluble antioxidant capacity and the ORAC/lipo is the lipid soluble antioxidant capacity. ORAC/total is the sum of ORAC/hydro and ORAC/lipo: Trolox, a water-soluble Vitamin E analog, is used as the calibration standard and the ORAC result is expressed as micromole Trolox equivalent (TE) per gram. Caffeic acid is used as the calibration standard and the HORAC (an ORAC test that is specific for hydroxyl radicals) result is expressed as μmole Caffeic acid equivalent (CAE) per gram. Trolox is used as the calibration standard and the NORAC result is expressed as μmole Trolox equivalent (TE) per gram.

Cyclooxygenase (COX) activity was measured at 37° C. by monitoring oxygen consumption using an Oxytherm Electrode Unit by Hansatech. IC50 is equal to the concentration of the sample that inhibits 50% of the activity of the enzyme under the assay conditions. Selective inhibitors of COX-2 relieve pain and inflammation.

Data from these various tests, as conducted by an independent laboratory, of a representative sample of an inventive composition with 40% Mangostin are reported in Table 5. A comparison of representative samples of 1%, 10%, 20%, and 40% Mangostin composition are detailed in Table 6. These in vitro test data demonstrate highly effective free radical scavenging activity by the inventive mangostin compositions against peroxyl, hydroxyl, peroxynitrite, and superoxide anion radical.

TABLE 5
ORAC and COX Test Data for a 40% Mangostin composition

| Parameter | Value | |
|---|---|---|
| ORAC/hydro | 878 | μmole TE/g |
| ORAC/lipo | 3,331 | μmole TE/g |
| ORAC/total | 4,209 | μmole TE/g |
| HORAC+ | 13 | μmole CAE/g |
| NORAC\hat{ } | 1 | μmole TE/g |
| COX-1 IC₅₀ | 0.046 | mg/ml |
| COX-2 IC₅₀ | 0.069 | mg/ml |

| | |
|---|---|
| COX-1 IC₅₀/COX-2 IC₅₀ | 0.667 |

TABLE 6
Comparison of ORAC Data for Various Mangostin Compositions

| Test Composition | ORAC/hydro | ORAC/lipo | ORAC/total |
|---|---|---|---|
| 1% Mangostin | 1,945 | 257 | 2,202 |
| 10% Mangostin | 1,940 | 1,174 | 3,114 |
| 20% Mangostin | 2,058 | 2,668 | 4,726 |
| 40% Mangostin | 878 | 3,331 | 4,209 |

Equivalents of the Invention

While a number of preferred embodiments of the invention and variations thereof have been described in detail, other modifications and methods of using the disclosed therapeutic combinations will be apparent to those of skill in the art. Accordingly, it should be understood that various applications, modifications, and substitutions may be made of equivalents without departing from the spirit of the invention or the scope of the claims. Various terms and conventions have been used in the description to convey an understanding of the invention. It will be understood that a corresponding description of these various terms applies to common linguistic or grammatical variations or forms of these various terms. It will also be understood that some compounds have been identified by trade names, but that these names are provided as contemporary examples, and the invention is not limited by such literal scope, particularly when compounds have been described in chemical terms. Although the written description offers biochemical theory and interpretation of available data in describing the invention, it should be understood that such theory and interpretation do not bind or limit the claims. Further, it should be understood that the invention is not limited to the embodiments set forth herein for purposes of exemplification, but is to be defined only by a fair reading of the appended claims, including the full range of equivalency to which each element thereof is entitled.

We claim:

1. A composition comprising a concentrated extract of mangosteen pericarp, wherein the extract includes xanthones at a concentration between about 0.1% to about 80% of the total weight of the composition, and wherein the composition includes tannins at a concentration that is modulated independently of the xanthone concentration.

2. The composition of claim 1, wherein the extract is present at a concentration between about 0.3% to about 60% of the total weight of the composition.

3. The composition of claim 1, wherein the extract is present at a concentration between about 1.0% to about 40% of the total weight of the composition.

4. The composition of claim 1, wherein the extract is present at a concentration of about 1% of the total weight of the composition.

5. The composition of claim 1, wherein the extract is present at a concentration of about 10% of the total weight of the composition.

6. The composition of claim 1, wherein the extract is present at a concentration of about 20% of the total weight of the composition.

7. The composition of claim 1, wherein the extract is present at a concentration of about 40% of the total weight of the composition.

8. The composition of claim 1, wherein the composition is a therapeutic agent sufficient for the prevention or treatment of disease.

9. The composition of claim 8, wherein the composition is a therapeutic agent sufficient for the prevention or treatment of human diseases.

10. The composition of claim 8, wherein the composition is sufficient for the prevention or treatment of diseases of the skin.

11. The composition of claim 10, wherein the diseases of the skin are associated with exposure to ultraviolet radiation.

12. The composition of claim 10, wherein the diseases of the skin are at least one selected from the group consisting of sunburn, photosensitivity, skin-localized allergy, immunosuppression, premature aging, psoriasis, cancerous lesions, precancerous lesions, bacterial infection, viral infection, fungal infection, and rash.

13. The composition of claim 12, wherein the precancerous lesion or cancerous lesion is one selected from the group consisting of actinic keratosis, basal cell cancer, squamous cell cancer, and malignant melanoma.

14. The composition of claim 10 wherein the disease of the skin is polymorphic light eruption.

15. The composition of claim 8, wherein the composition is sufficient for bioprotection of tissues, wherein such bioprotection prevents or treats conditions selected from the group consisting of allergy, inflammation, and apoptosis.

16. The composition of claim 8, wherein the composition is an antioxidant that is sufficient for treatment of diseases that are a consequence of exposure to reactive oxygen species.

17. The composition of claim 16, wherein the diseases that are a consequence of exposure to reactive oxygen species are skin diseases.

18. A composition comprising a concentrated extract of mangosteen pericarp, wherein the extract comprises at least one of the xanthones selected from the group consisting of α-mangostin, β-mangostin, and γ-mangostin, and wherein the composition includes tannins at a concentration that is modulated independently of the xanthone concentration.

19. The composition of claim 18 further comprising at least one of the xanthones selected from the group consisting of calabaxanthone, demethylcalabaxanthone, 6-deoxy-γ-mangostin, 1-isomangostin, 3-isomangostin, 1-isomangostin hydrate, 3-isomangostin hydrate, gartanin, 8-deoxygartanin, garcinone A, garcinone B, garcinone C, garcinone D, garcinone E, mangostanol (prenyl xanthone), mangostanol (polyoxygenated xanthone), 6-deoxy-γ-mangostin, mangostinone, 1,5-dihydroxy-2-(3-methylbut-2-enyl)-3-methoxyxanthone, 1,7-dihydroxy-2-(3-methylbut-2-enyl)-3-methoxyxanthone, 1,5-dihydroxy-3-methoxy-2-(3-methylbut-2-enyl)xanthone, 1,7-dihydroxy-3-methoxy-2-(3-methylbut-2-enyl)xanthone, 5,9-dihydroxy-2,2-dimethyl-8-methoxy-7-(3-methylbut-2-enyl)-2H,6H-pyrano[3,2b]xanthen-6-one, 2-(γ,γ-dimethylallyl)-1,7-dihydroxy-3-methoxyxanthone, 2,7-di-(3-methylbut-2-enyl)-1,3,8-trihydroxy-4-methylxanthone, 2,8-Di-(3-methylbut-2-enyl)-7-carboxy-1,3-dihydroxyxanthone, normangostin (ν-mangostin), 1,5,8-trihydroxy-3-methoxy-2-(3-methyl-2-butenyl)xanthone, 1,7-dihydroxy-2-isoprenyl-3-methoxyxanthone, xanthone I, BR-xanthone A, BR-xanthone B (2,4,5-trihydroxy-1-methoxyxanthone), garcinone B, mangostanol, mangostenol, mangostenone A, mangostenone B, tovophyllin, and trapezifolixanthone.

20. The composition of claims 18 or 19, further comprising one or more compounds of the group consisting of (−)-epicatechin, procyanidin A-2, procyanidin B-2, garcinone A, garcinone B, garcinone C, garcinone D, garcinone E, and maclurin.

21. A composition comprising at least one of the xanthones selected from the group consisting of α-mangostin, β-mangostin, and γ-mangostin, and wherein the composition includes tannins at a concentration that is modulated independently of the xanthone concentration.

22. The composition of claim 21 further comprising at least one of the xanthones selected from the group consisting of calabaxanthone, demethylcalabaxanthone, 6-deoxy-γ-mangostin, 1-isomangostin, 3-isomangostin, 1-isomangostin hydrate, 3-isomangostin hydrate, gartanin, 8-deoxygartanin, garcinone A, garcinone B, garcinone C, garcinone D, garcinone E, mangostanol (prenyl xanthone), mangostanol (polyoxygenated xanthone), 6-deoxy-γ-mangostin, mangostinone, 1,5-dihydroxy-2-(3-methylbut-2-enyl)-3-methoxyxanthone, 1,7-dihydroxy-2-(3-methylbut-2-enyl)-3-methoxyxanthone, 1,5-dihydroxy-3-methoxy-2-(3-methylbut-2-enyl)xanthone, 1,7-dihydroxy-3-methoxy-2-(3-methylbut-2-enyl)xanthone, 5,9-dihydroxy-2,2-dimethyl-8-methoxy-7-(3-methylbut-2-enyl)-2H,6H-pyrano[3,2b]xanthen-6-one, 2-(γ, γ-dimethylallyl)-1,7-dihydroxy-3-methoxyxanthone, 2,7-di-(3-methylbut-2-enyl)-1,3,8-trihydroxy-4-methylxanthone, 2,8-Di-(3-methylbut-2-enyl)-7-carboxy-1,3-dihydroxyxanthone, normangostin (ν-mangostin), 1,5,8-trihydroxy-3-methoxy-2-(3-methyl-2-butenyl)xanthone, 1,7-dihydroxy-2-isoprenyl-3-methoxyxanthone, xanthone 1, BR-xanthone A, BR-xanthone B (2,4,5-trihydroxy-1-methoxyxanthone), garcinone B, mangostanol, mangostenol, mangostenone A, mangostenone B, tovophyllin, and trapezifolixanthone.

23. The composition of claims 21 or 22, further comprising one or more compounds of the group consisting of (−)-epicatechin, procyanidin A-2, procyanidin B-2, garcinone A, garcinone B, garcinone C, garcinone D, garcinone E, and maclurin.

24. The composition of claim 1, wherein the composition is a nutritional supplement, sufficient to provide healthful benefit.

25. The composition of claim 24, wherein the nutritional supplement is incorporated into a form selected from the group consisting of food and beverage.

26. The composition of claim 8, further comprising at least one second therapeutic agent to form a combination therapeutic entity, wherein the entity is sufficient to prevent or treat disease.

27. The combination therapeutic entity of claim 26, wherein the entity is sufficient to prevent or treat a disease of the skin.

28. The composition of claim 27, wherein said disease of the skin includes one or more conditions selected from the group consisting of abnormal cutaneous differentiation, proliferation, or pigmentation, bacterial infections, parasitic infections, fungal infections, inflammation, pain or irritation from any source, pruritis, viral agents, keratolysis, UV radiation damage, seborrhea, dandruff, and acne.

29. The composition of claim 27, wherein the at least one second agent is selected from the group consisting of modifiers of cutaneous differentiation and/or proliferation and/or pigmentation, antibacterial agents, antiparasitic agents, antifungal agents, steroidal anti-inflammatory agents, anaesthetic agents, antipruriginous agents, antiviral agents, keratolytic agents, other anti-oxidants, antiseborrhoeic agents, antidandruff agents, and antiacne agents.

30. A method for treating diseases of the skin caused by exposure to sunlight, the method comprising administering a composition comprising a concentrated extract of mangosteen pericarp, wherein the extract is at a concentration between about 0.1% to about 80% of the total weight of the composition, and wherein such composition has an independently modulated tannin concentration.

31. The method of claim 30, wherein the extract is particularly at a concentration of about 0.3% to about 60% of the total weight of the composition.

32. The method of claim 30, wherein the extract is more particularly at a concentration of about 0.1% to about 40% of the total weight of the composition.

33. A process (A.1), wherein rind is water-extracted, and the thereby spent rind is subsequently extracted with organic solvent and again with water, comprising:
1. charging a volume of mangosteen rind pieces into an extractor,
2. charging about 6 volume of water into the extractor,
3. heating the extractor to about 85° C. and maintaining the temperature up to about 2 hour,
4. cooling the extractor to room temperature,
5. filtering the water extract into a container to yield water extract # 1,
6. charging 4 volume of water into the extractor,
7. heating the reactor to about 85° C. and maintaining the temperature up to about 2 hours,
8. cooling the reactor to room temperature,
9. filtering the water extract into container to yield water extract # 2, and
10. combining both the water extracts #1 and #2, and concentrating to about 20-25% of total solids, to yield as extract-a, which is a part of product # I.

34. A process (A.2), wherein the spent rind of process A.1 is extracted with organic solvent, and again with water, comprising:
1. charging a volume of re-dried spent of mangosteen rind from process A,1, in to a extractor,
2. charging about 6 volume of about 80% alcohol into the reactor,
3. heating of the reactor up to 65° C.-75° C. and maintaining the temperature at reflux condition of the extracting solvent for up to about 2 hours,
4. cooling the reactor to room temperature,
5. filtering the alcohol extract into a container to yield solvent extract # 1,
6. charging about 4 volume of alcohol into the reactor,
7. heating the reactor up to reflux temperature and continuing the reflux up to about 2 hours,
8. cooling the reactor to room temperature,
9. filtering the extract into a container to yield solvent extract # 2,
10. repeating the steps 6 to 9 and collecting the extract (solvent extract # 3),
11. combining all 3 solvent extracts and charging into a reactor,
12. distilling off the solvent from the extract and concentrating to a semi solid paste containing about 40-45% total solids,
13. holding the semi solid paste in room temperature for 8-10 hours for settling,
14. dividing the paste, after settling, into two parts, an upper layer and a lower layer,
15. separating the lower layer and filter through 5-micron filter,
16. mixing the lower layer and the residue after filtering the upper layer to give a wet cake,
17. drying the wet cake under constant vacuum at about 75-80° C. and pulverizing it to yield extract-b, (this product is a part of product # ii,) and
18. concentrating the filtered upper layer to a paste of about 20-25% of total solids, (this product is highly water soluble and added in to extract-a, which is a part of product # I).

35. A process (A.3) wherein the spent after solvent extraction is once again extracted with water to obtain other tannins, the process comprising,
1. charging a volume of dried spent into a extractor,
2. charging about 4 volume of water into the reactor,
3. heating of the reactor up to 85° C. and maintaining the temperature up to 2 hours,
4. cooling the reactor to room temperature,
5. filtering the water extract into a container to yield water extract # 1,
6. charging about 4 volume of water in to the reactor,
7. heating the reactor to 85° C. and maintaining the temperature up to about 2 hours,
8. cooling the reactor to room temperature,
9. filtering the water extract in to a container, to yield water extract # 2, and
10. combining both the extracts and concentrating to about 20-25% of total solids, (this water extract is added into extract-a of process A.1, which is a part of product # I).

36. A process (A.4), wherein the wet cake of extract-b of process A.2, is again processed to obtain an extract containing at least 40% mangostin, which is a part of product # III, the process comprising:
1. charging a volume of the extract (wet cake) containing 20% mangostin in a reactor,
2. adding 8 volume of water in to the reactor,
3. stirring well for about 30 minutes,
4. heating the reaction mass to 75-80° C. for 1 hour under constant stirring,
5. cooling the reaction mass to room temperature,
6. unloading the reaction mass into a container,
7. holding the reaction mass in room temperature for about 10-12 hours,
8. decanting the top layer and collecting the bottom sediment mass,
9. filtering the sediment mass and collecting the wet cake,
10. adding water to the wet cake and making it in to a slurry,
11. filtering to remove tannins from the product,
12. repeating the water washings twice more,
13. drying the wet cake under vacuum at about 75-80° C. and pulverizing it (extract-c); this product is a part of product # III,
14. combining all the three water washings and charging into a reactor, and
15. concentrating the material to a paste of 20-25% of total solids; this water extract is added into extract-a of process A. 1, which is a part of product # I).

37. A process (method B.1) wherein the rind is extracted first with organic solvent and then with water, the process comprising
1. charging a volume of dried mangosteen rind in to a extractor,
2. charging 6 volume of about 80% alcohol,
3. heating of the reactor to about 65° C.-75° C. and maintaining the temperature at reflux condition of the extracting solvent and continuing up to 2 hours,
4. cooling the reactor to room temperature,
5. filtering the alcohol extract into a container (solvent extract # 1),
6. charging about 4 volume of alcohol into the reactor,
7. heating of the reactor up to reflux temperature and continuing the reflux up to about 2 hours,
8. cooling the reactor to room temperature,
9. filtering the extract into a container, (solvent extract # 2),
10. repeating the steps 5 to 9 and collecting the extract (solvent extract # 3),
11. combining all the 3 solvents extracts and charging it in a suitable reactor,
12. distilling off the solvent from the extract and concentrating it to a semi solid paste containing about 40-45% total solids,
13. holding the paste for about 10 hours,
14. filtering the paste,
15. drying the wet cake in vacuum at about 75-80° C. and pulverizing it (extract-b), both extract-b of process # a, 2 and extract-b of process # b, 1 are combined and this product is a part of product # II, (product-I may be added to product 11 to equilibrate the content of mangostin, flavonoids and tannins to give the finished product II), and
16. adding the filtrate into extract-a of process A.1, which is a water soluble product and is included in the product-I.

38. A process (B.2), wherein the spent rind of process B.1 is extracted with water, the process comprising:
1. charging a volume of dried spent from process B.1 into an extractor,
2. charging about 6 volume of water into the extractor,
3. heating of the extractor to about 85° C. and maintaining the temperature up to about 2 hours,
4. cooling the reactor to room temperature,
5. filtering the water extract into a container, (water extract # 1),
6. charging 4 volume of water into the extractor,
7. heating of the extractor to 85° C. and maintaining temperature up to about 2 hours,
8. cooling the reactor to room temperature,
9. filtering the water extract in to a container to yield water extract # 2,
10. repeating the steps 6 to 9 and collecting the extract to yield water extract # 3,
11. combining all the 3 water extracts and charging it into a reactor,
12. distilling off the water in the extract and concentrating to about 20-25% of total solids, and
13. adding this extract to the water extract-a of process A.1, which is a part of product I.

39. A process (B.3) for enriching the extract containing 20% mangostin to 40% mangostin, wherein the wet cake obtained during the process # B. 1 is further washed with water, the process comprising
1. charging a volume of the extract containing 20% γ-mangostin in a suitable reactor,
2. adding 8 volume of water in to the reactor, and stirring well for about 30 minutes,
3. heating the reaction mass to about 75-80° C. for 1 hour under constant stirring,
4. cooling the reaction mass to room temperature,
5. unloading the reaction mass in a container,
6. holding the reaction mass in room temperature for about 10-12 hours,
7. decanting the top layer and collecting the bottom sediment mass,
8. filtering the sediment mass and collecting the wet cake,
9. adding water to the wet cake and making it into a slurry,
10. filtering the slurry to remove tannins from the product,
11. repeating the water washings 2 more times,
12. drying the wet cake under vacuum at about 75-80° C. and pulverizing it (extract-c), both extract-c of process a, 4 and the extract-c of process b, 3 are combined and this product is used for the production of product # III, (product-I and/or product-II may be added to product III to equilibrate the content of γ-mangostin, flavonoids and tannins to give the finished product III as described in table 1,) the chemical composition of the product is as follows:
combining all the three water washings and charging into a reactor, and
14. concentrating the combined washings to a paste of 20-25% of total solids.

40. A process for deriving a 40% mangostin extract comprising:
1. after extracting the mangosteen rind pieces with water (ref: process # 1), the residual water from the spent is completely drained off, the remaining moistened mangosteen rind pieces is known as mangosteen spent,
2. charging known quantity of mangosteen spent in to a suitable extractor,
3. charging 6 volume of 90% organic solvent,
4. initiating the heating if the reactor by passing the steam into the outer jacket,
5. heating to 65° C.-75°c and maintain temperature at reflux condition of the extracting solvent and continuing up to 2 hours,
6. ceasing heat input after 2 hours, and cooling to room temperature,
7. filtering the alcohol extract in to a well cleaned stainless steel container, (solvent extract # 1)
8. charging 4 volume of alcohol into the extractor,
9. initiating the heating of the reactor by passing the steam in to the outer jacket,
10. heating up to reflux temperature and continuing the reflux up to 2 hours,
11. ceasing the heating after 2 hours, and cooling to room temperature,
12. filtering the extract in to a well cleaned stainless steel container, (solvent extract # 2)
13. repeating the steps 7 to 11 and collect the extract (solvent extract # 3),
14. combining all the 3 solvent extracts and collect it in a suitable reactor,
15. distilling off the solvent from the extract and concentrate to a semi solid paste containing 40-45% total solids,
16. holding concentrated paste at room temperature for about 10-12 hour for sedimenting,
17. dividing the paste into upper and lower layers, both layers are separated and analyzed,
18. separating the lower layer and filtering it,
19. adding the minimum amount of water and making it in to a slurry,
20. filtering through 5-micron filter and sucking the wet cake dry, and
21. drying the wet cake under constant vacuum at 80° C. to yield the final product, a light brown powder.

41. A process (method 2) for deriving an extract of 40% mangostin, the process comprising:
1. charging a volume of the extract containing =20% mangostin into a reactor,
2. adding 5 volume of water into the reactor,
3. stirring well for 30 minutes,
4. adding a 5% KOH solution to the reaction mass slowly under constant stirring to bring the ph to 8, 0 to 8, 2,
5. heating the reaction mass to 55-60° C. for 1 hour under constant stirring,
6. cooling the reaction mass to room temperature,
7. checking the ph and maintain the pH 8, 0 to 8, 2 by adding 5% KOH,
8. heating the reaction mass to 55-60° C. for 0.5 hour under constant stirring,
9. cooling the reaction mass to room temperature,
10. unloading the reaction mass in a container,
11. holding the reaction mass in room temperature for 10-12 hours,
12. decanting the top layer and collecting the bottom sediment mass,
13. filtering the sediment mass through 5 micron filter and collecting the wet cake,
14. adding minimum amount of water to the wet cake to making it in to a slurry,
15. filtering the slurry to remove excess KOH from the product, and
16. drying the wet cake under vacuum at 75° C.

42. A process (# 2 of method 2) for extracting 40% mangostin, the method comprising:
1. charging a volume of the extract containing 20% mangostin in a reactor,
2. adding 8 volume of water in to the reactor,
3. stirring well for 30 minutes,
4. heating the reaction mass to 75-80° C. for 1 hour under constant stirring,
5. cooling the reaction mass to room temperature, and then chilling the reaction mass to −5° C. for 5 hour,
6. allowing the reaction mass to return to room temperature,
7. unloading the reaction mass into a container, and holding at room temperature for about 10-12 hours,
8. decanting the top layer and collecting the bottom sediment mass,
9. filtering the sediment mass and collecting the wet cake,
10. adding water to the wet cake and making it into slurry,
11. filtering the slurry to remove tannins from the product,
12. repeating the water washings 2 more times,
13. drying the wet cake under vacuum at 75° C. and pulverizing it, combining all the three water washings and charged into a reactor,
14. concentrating it to a paste of 20-25% total solids, and
15. spray-drying the product.

43. A method for preparing a concentrated extract of mangosteen pericarp comprising;
operating a process according to claim 33,
operating a process according to claim 34,
operating a process according to claim 35,
operating a process according to claim 36,
operating a process according to claim 37,
operating a process according to claim 38, and
operating a process according to claim 39.

44. The method according to claim 43 wherein the final product has a content of at least 1%, a flavonoid content of at least 5%, and tannins of at least 15%.

45. The method according to claim 43, wherein the final product has a content of at least 20%, a flavonoid content of at least 40%, and tannins of at least 12%.

46. The method according to claim 43, wherein the final product has a content of at least 40%, and a flavonoid content of at least 95%.
